(19) United States
(12) Patent Application Publication
(10) Pub. No.: US 2026/0199349 A1
(43) Pub. Date: Jul. 16, 2026

(54) Treatment of Cancers Having Mutations in WNT Pathway Tumour Suppressors

(71) Applicant: RECURSION PHARMACEUTICALS, INC., Salt Lake City, UT (US)

(72) Inventor: Ashish Ashok Bhandari, Cincinnati, OH (US)

(21) Appl. No.: 19/119,135

(22) PCT Filed: Oct. 20, 2023
(86) PCT No.: PCT/US2023/035576

§ 371 Date: Apr. 8, 2025

Related U.S. Application Data
(60) Provisional application No. 63/418,104, filed on Oct. 21, 2022.

(51) Int. Cl. A61K 31/519; A61K 45/06; A61P 35/00

(52) U.S. Cl. CPC A61K 31/519; A61K 45/06; A61P 35/00

(57) ABSTRACT

A method of treating a cancer having one or more mutations in tumour suppressors AXIN1 and/or APC in the WNT pathway in a subject in need thereof is provided wherein the method comprises administering a MEK inhibitor or a pharmaceutically acceptable salt thereof to the subject. The MEK inhibitor may be administered as a single agent or may be combined with treatment with an immune checkpoint inhibitor, such as an inhibitor of PD-1 and/or PD-L1, or a RAF inhibitor.

### FIELD

The present disclosure relates generally to the use of mutations in predicting a patient's response to an anti-proliferative agent, specifically, a MEK inhibitor. In particular, the presence or absence of mutations in the WNT pathway, specifically in tumour suppressors AXIN1 and/or APC, can be used to predict a response to treatment with a MEK inhibitor, either as a single agent or in combination with other RAS/MAPK pathway inhibitors and/or immune checkpoint inhibitors in a patient presenting with cancer. The present disclosure thus provides a method of treating a specific subset of cancer patients, wherein the patients are identified as having mutations in tumour suppressors AXIN1 and/or APC in the WNT pathway, using MEK inhibitors. This treatment may be as a single agent or may be combined with other RAS/MAPK pathway inhibitors and/or immune checkpoint inhibitors, such as an inhibitor of PD-1 and/or PD-L1.

### BACKGROUND

WNT signalling is involved in several physiological processes including embryonic development, stem cell homeostasis, tissue regeneration, and lineage commitment. Aberrant activation of the WNT pathway through either gain-of-function or loss-of-function mutations appears frequent across a wide variety of human cancers. Central to the pathogenesis of WNT altered tumours is the proteolytic turnover of β-catenin, which functions as a transcriptional co-activator of WNT target genes. In particular, the levels of β-catenin are kept low by a multisubunit destruction complex composed of the tumour suppressors AXIN1 and adenomatous polyposis coli (APC) and the kinases casein kinase 1 (CK1) and glycogen synthase kinase 3β (GSK3β). Inactivating mutations in AXIN1 and APC are frequently observed across many cancers including hepatocellular carcinoma (HCC), colorectal, liver, bladder, endometrial, melanoma, ovarian, lung, pancreatic, and gastric cancers. Tumours harbouring loss-of-function mutations in AXIN1 and APC are often clinically aggressive and less sensitive to treatments with chemotherapies and/or immunotherapies. For example, retrospective analysis in AXIN1 mutant advanced hepatocellular carcinoma (HCC) patients treated with the combination of atezolizumab and bevacizumab had less favourable responses to the treatment compared to with the non-AXIN1 mutant population (Zhu, A. X., et al. Nat Med., 2022, 28:1599-1611). Accordingly, there is a substantial need for developing therapeutics that can overcome mutant induced WNT activation, specifically for the treatment of AXIN1 and APC mutant cancers as these mutations are often considered undruggable (Parsons, M J, et al. Cancer Discov., 2021, 11 (10): 2413-2429. Bugter, J. M., et al. Nat Rev Cancer., 2021, 5-21).

Hepatocellular carcinoma (HCC) is one example of a cancer wherein loss-of-function mutations in AXIN1 and/or APC may be present. HCC is the third leading cause of cancer-related deaths worldwide. Patients with early-stage HCC can be treated successfully with surgical resection or liver transplantation. However, the usual late diagnosis of HCC precludes curative treatments, and systemic therapies are the only viable option for inoperable patients. Current possible treatments for advanced HCC are sorafenib, lenvatinib, a combination of atezolizumab and bevacizumab, regorafenib, ramucirumab, cabozantinib, nivolumab and pembrolizumab. Sorafenib is an orally available multikinase inhibitor approved as a systemic therapy for treating patients with advanced HCC. The benefits provided by sorafenib have been shown to be limited and new drugs are being developed to overcome sorafenib resistance and improve patients' prognoses, for example, the c-MET inhibitor cabozantinib. However, the severity of HCC, the lack of good diagnostic markers and treatment strategies, and clinical heterogeneity make management of the disease a major challenge. Further difficulties arise due to long median times to responses for checkpoint therapies in the frontline (~12 weeks), a median overall survival benefit of <6 months and limited treatment options for patients with Child Pugh B/C classifications.

Patients with HCC have a highly variable clinical course indicating that HCC comprises several biologically distinctive subgroups. Telomerase reverse transcriptase (TERT) promoters have been found to be mutated in more than 50% of HCC tissue samples examined, making them the most frequently occurring single-nucleotide mutations observed in HCC. Tumour protein 53 (TP53) is the second most frequently mutated gene in HCC, occurring in more than 30% of cases of HCC. Catenin beta 1 (CTNNB1) is another one of most frequently mutated genes in HCC and aberrant activation of β-catenin has been observed in 20-30% of HCC patients. About 40% of HCC patients harbour mutations in the WNT pathway. AXIN1 is the second most frequently mutated gene in this pathway. Loss of AXIN1 is found in approximately 11% of patients with HCC and is considered to confer innate resistance to immune-checkpoint blockade. Despite belonging to the same pathway, genetic alterations in CTNNB1 and AXIN1 are found to be mutually exclusive.

Similarly, mutations of CTNNB1, AXIN and/or APC have been observed in epithelial ovarian cancer (EOC), particularly in the endometrioid and mucinous subtypes of EOC. EOC is the deadliest female malignancy. Aberrant activation of the WNT pathway in EOC leads to the hyperactivity of β-catenin.

Colorectal cancer (CRC) is one of the most common types of cancer in the world. CRC has recently been further classified into various molecular subtypes, called consensus molecular subtypes (CMS). CMS classifications are not used to drive treatment decisions today, but are used to distinguish biological, clinical, and molecular features of CRC, creating awareness for clinicians around the heterogeneity of CRC. For example, on CMS subtype, CMS2, is called canonical and is marked by WNT and MYC signalling activation. APC mutant CRC tumours are found more predominantly enriched in CMS2 CRCs and tend to harbour co-occurring KRAS and TP53 alterations (Bugter, J. M., et al. Nat Rev Cancer., 2021, 5-21).

The RAS/MAPK pathway is an important signal transmission pathway in a cell and plays a significant role in proliferation and differentiation processes. Growth factor-induced signals are transmitted by successive phosphorylation from the serine/threonine kinase Raf to the dual-specific kinase MEK (MAP kinase kinase/ERK kinase) and finally to the kinase ERK (extracellular signal regulated kinase), thus influencing gene expression.

Currently, the use of MEK inhibitors for therapeutic treatment of cancers is restricted to treatment of BRAF mutant populations including melanoma and non-small cell lung cancer (NSCLC) wherein treatment is combined with RAF inhibitors. MEK inhibitors are administered to patients harbouring BRAF V600E mutations only in combination with RAF inhibitors, and not as a single agent, due to pathway specific rebound with MEK inhibitors in these patients. Additionally, the MEK inhibitor selumetinib is also approved for the genetic indication neurofibromatosis type 1 (NF1). However, outside of cancers harbouring RAS/MAPK pathway alterations, the use of MEK inhibitors alone or in combination for genetically defined tumours has not been formally investigated. In fact, in CRC, MEK inhibition has been shown to activate WNT signalling genetically and pharmacologically as suggested by increases in AXIN2 expression levels upon siRNA KD in the APC WT CRC cell line HCT116. However, in a colony forming assay, single agent trametinib was able to reduce viability of HCT116 cells in a dose dependent manner (Zhan et al., Nat Comm, 2019), suggesting that pharmacological induction of WNT activation is not sufficient to drive resistance. A separate group reported that single agent MEK inhibition alone did not significantly alter AXIN2 expression levels or WNT activity in the more relevant APC mutant CRC cell line COLO320 (Solberg et al., Cancers, 2019). Fully elucidating the context specificity between WNT signalling and RAS/MAPK pathway inhibitors may expand the therapeutic use of MEK inhibitors.

### SUMMARY

According to a first aspect, there is provided a method of treating a cancer having one or more mutations in AXIN1 and/or APC in a subject in need thereof, the method comprising administering a MEK inhibitor or a pharmaceutically acceptable salt thereof to the subject.

According to a second aspect, there is provided a method of using one or more mutations in AXIN1 and/or APC in a subject suffering from cancer as a biomarker to evaluate the likelihood that a MEK inhibitor or a pharmaceutically acceptable salt thereof would produce an anti-cancer effect in the subject, the method comprising assaying for the presence of one or more mutations in AXIN1 and/or APC in the subject; and if one or more mutations in AXIN1 and/or APC are present in the subject, administering a MEK inhibitor or a pharmaceutically acceptable salt thereof to the subject to produce the anti-cancer effect.

According to a third aspect, there is provided a MEK inhibitor or a pharmaceutically acceptable salt thereof for use in treatment of a cancer having one or more mutations in AXIN1 and/or APC in a subject in need thereof.

### BRIEF DESCRIPTION OF THE FIGURES

The disclosure will be more clearly understood from the following description of some embodiments thereof, given by way of example only, with reference to the following figures in which:

FIG. 1 illustrates tumour volume results for treatment with MEK inhibitor REC-4881 versus Cabozantinib in the HCC AXIN1 mutant LI6612 PDX model;

FIG. 2 illustrates tumour volume results for treatment with MEK inhibitor REC-4881 and four FDA approved MEK inhibitors in the HCC AXIN1 mutant LI6612 PDX model;

FIG. 3 illustrates tumour volume for treatment with MEK inhibitor REC-4881 at 3 mg/kg PO single agent or in combination with anti-PD-L1 at 10 mg/kg IP in the B16F10-ova syngeneic melanoma model which harbours an APC mutation;

FIG. 4A shows Tumour Growth Inhibition (TGI), and FIG. 4B shows probability of Progression Free Survival (PFS) results with MEK inhibitor REC-4881 at 3 mg/kg PO across 19 HCC PDX models run as a mouse clinical trial where 6 of the models harbour AXIN1 mutations and 13 of the models do not harbour AXIN1 mutations;

FIG. 5A shows Tumour Growth Inhibition (TGI) and FIG. 5B shows probability of Progression Free Survival (PFS) results with MEK inhibitor REC-4881 at 3 mg/kg PO across 10 Ovarian PDX models run as a mouse clinical trial where 5 of the models harbour AXIN1 and/or APC mutations and 5 of the models do not harbour AXIN1 and/or APC mutations;

FIG. 6A shows Tumour Growth Inhibition (TGI) and FIG. 6B shows probability of Progression Free Survival (PFS) results with MEK inhibitor REC-4881 at 3 mg/kg PO across the combined studies of the 19 HCC PDX models and 10 Ovarian PDX models run as a mouse clinical trial;

FIG. 7 shows tumour volume results for treatment with MEK inhibitor REC-4881 at 1 mg/kg and 3 mg/kg PO versus sorafenib in the HCC LI6692 PDX model, which harbours an AXIN1 mutation;

FIG. 8 illustrates pharmacodynamic markers from tumour samples harvested from the in vivo PDX study of Example 7, FIG. 8A for pERK/ERK; FIG. 8B for pMEK/MEK; FIG. 8C for SPRY4; FIG. 8D for DUSP6; and FIG. 8E for PPIA;

FIG. 9 illustrates pharmacokinetic data from non-tumour bearing NCG mice administered REC-4881 at 1 mg/kg and 3 mg/kg;

FIG. 10 shows viability curves for human colorectal cancer cell lines, APC mutant and wild-type, treated with MEK inhibitor REC-4881 for 72 hours; and

FIG. 11A shows REC-4881 modulates CDKN2A expression and FIG. 11B shows REC-4881 modulates MYC expression in APC mutant and wild-type colorectal cancer cell lines.

FIG. 12 shows CD8⁺ T cells (A) and Tregs (B) effects in a APC Mutant B16F10-ova Murine Melanoma Tumor in Female C57BL/6 Mice administered vehicle, an anti-PD1 agent, REC-4881 (3 mg/kg) or a combination of the two.

FIG. 13 shows study on the effect of REC-4881 alone or with an anti-PD1 agent in AXIN1-null Hepa 1-6 Murine Hepatocellular Carcinoma (HCC) tumors in Female C57BL/6 mice, (A) dosing schedule; (B) tumor volume measured for each arm of the study; (C) showing the HCC model was engineered to knock out AXIN1; (D) results at Day 22 and Day 24.

### DETAILED DESCRIPTION

Provided herein is a method of treating a specific subset of cancers, that is cancers having one or more mutations in AXIN1 and/or APC tumour suppressors in the WNT pathway, by administering a MEK inhibitor to a subject. This subset of cancers are often clinically aggressive and less sensitive to treatments with chemotherapies and/or immunotherapies. As shown herein, treatment with a MEK inhibitor results in a favourable progression free survival benefit for models harbouring AXIN1 and/or APC mutations compared to models without AXIN1 and/or APC mutations. The present disclosure thus provides a targeted treatment for this specific subset of cancers. Targeting treatment to this specific subset of cancers will help result in the most optimal, patient-tailored treatment to maximize treatment response, prolong survival, minimize the treatment cost and avoid potential unwanted adverse effects of ineffective therapy.

The method comprises administering a MEK inhibitor or a pharmaceutically acceptable salt thereof to the subject having one or more mutations in AXIN1 and/or APC. The present disclosure thus provides a MEK inhibitor or a pharmaceutically acceptable salt thereof for use in the treatment of these subjects. It is hypothesised that MEK inhibition may restore function of AXIN1 and/or APC and dampen WNT driven growth of a mutated tumour. This is surprising as it has previously been reported that MEK inhibition in colorectal cancer downregulates AXIN1, thus increasing rather than decreasing WNT signalling.

The treatment with a MEK inhibitor may be combined with one or more additional treatments, such as treatment with an immune checkpoint inhibitor. In this respect, it is hypothesised that MEK inhibition may sensitise AXIN1 and/or APC mutated tumours to treatment with an immune checkpoint inhibitor, such as an inhibitor of PD-1 and/or PD-L1. This may provide a treatment for cancers that were previously unresponsive to treatment with immune checkpoint inhibitors and/or allow for a lower effective dose of immune checkpoint inhibitors.

### MEK Inhibitors

The term MEK refers to a MAP kinase kinase/ERK kinase (MEK), which is part of the Raf/MEK/ERK kinase or RAS/MAPK signal transmission pathway. MEK phosphorylates and activates MAPK. MEK proteins are coded by seven different genes, among which MEK1 and MEK2 are of greatest significance. A MEK inhibitor is understood herein to refer to an inhibitor of MEK, that is, any compound that downregulates, reduces or ceases MEK activity and/or function. MEK inhibitors for use in the present disclosure preferably inhibit MEK 1/2 of a subject. The MEK inhibitor for use in the present disclosure may be a dual inhibitor—in that case, the MEK inhibitor may not only inhibit a MEK, preferably MEK1/2, but also its upstream kinase (i.e. MAPKKK). MEK 1/2 is the MAPKK in the Ras/Raf pathway, whereby Ras/Raf acts as MAPKKK and ERK1/2 acts as MAPK. An example of such a dual inhibitor for use in the present disclosure is PLX-4032. The term “MEK inhibitor” as used herein is understood to encompass pharmaceutically acceptable salts thereof. The term “MEK inhibitor” as used herein may refer to one MEK inhibitor or a combination of two or more MEK inhibitors.

The MEK inhibitor may be a MEK1/2 inhibitor or a pharmaceutically acceptable salt thereof. The MEK inhibitor may be an allosteric inhibitor. The MEK inhibitor may be a selective allosteric inhibitor of MEK1 and MEK2 (MEK1/2). The MEK inhibitor may be REC-4881 (TAK-733 or REC-2029 or REC-4881) or a pharmaceutically acceptable salt thereof. TAK-733 is an example of a selective, allosteric MEK1/2 inhibitor. The MEK inhibitor may be selected from the group: Binimetinib (MEK162, ARRY-438162, ARRY-162), Cobimetinib (GDC-0973, XL-518, RG7421), Selumetinib (AZD6244, ARRY-142,886), Trametinib (GSK1120212, JTP-74057), CI-1040, Mirdametinib (PD0325901), RO5126766 (CH5126766), RO4987655 (CH4987655), Refametinib (RDEA119, BAY 869766) and Pimasertib (MSC1936369, AS703026), or a pharmaceutically acceptable salt thereof. The MEK inhibitor may be selected from the group: PD98059, PD184352 (2-(2-chloro-4-iodo-phenylamino)-N-cyclopropylmethoxy-3,4-difluoro-benzamide), AZD8330, RDEA-1 19 (BAY-869766), AS703026 and PLX-4032 (Zelboraf® (Vemurafenib)), or a pharmaceutically acceptable salt thereof. The MEK inhibitor may be selected from the group: REC-4881, Binimetinib, Cobimetinib, Trametinib and Selumetinib, or a pharmaceutically acceptable salt thereof. The MEK inhibitor may be Binimetinib. The MEK inhibitor PD98059 inhibits the activation of MEK by the kinase Raf. The MEK inhibitor R05126766 is a protein kinase inhibitor specific for the Raf and MEK mitogen-activated protein kinases (MAPKs) with potential anti-neoplastic activity. Raf/MEK dual kinase inhibitor R05126766 specifically inhibits the kinase activities of Raf and MEK, resulting in the inhibition of target gene transcription that promotes malignant transformation of cells. The MEK inhibitor AS703026 is a highly selective and potent allosteric inhibitor of MEK 1/2.

A Cancer Having One or More Mutations in AXIN1 and/or APC (AXIN1 and/or APC Mutated Cancer)

The terms an “AXIN1 mutated cancer” and/or an “APC mutated cancer” are understood to refer to cancer in a subject wherein cells of the cancer contain one or more mutations in AXIN1 and/or APC. AXIN1 and APC are tumour suppressors in the WNT pathway. The one or more mutations may result in a loss of AXIN1 and/or APC function. AXIN1 and APC mutated cancers form a subset of WNT driven cancers. The one or more mutations may be mutations in AXIN1. The one or more mutations may be mutations in APC. The one or more mutations may be mutations in AXIN1 and APC. The one or more mutations may be somatic mutations and/or germline mutations. The mutations may be in the AXIN1 and/or APC genes. The one or more mutations may be truncations. The cancer may further comprise one or more mutations present in the TP53 gene. These mutations may result in a loss of TP53 function. The cancer may further comprise one or more mutations in BRAF. Alternatively, the cancer may comprise no mutations in BRAF.

The cancer having the mutations as described above may be selected from the group: hepatocellular carcinoma (HCC), colorectal, liver, bladder, endometrial, melanoma, ovarian, lung, pancreatic, and gastric cancers. The cancer may be HCC, for example, AXIN1 mutated HCC and/or APC mutated HCC. The cancer may be ovarian cancer, for example, AXIN1 mutated ovarian cancer and/or APC mutated ovarian, in particular, epithelial ovarian cancer (EOC), particularly in endometrioid and mucinous subtypes of EOC. The cancer may be melanoma, for example, AXIN1 mutated melanoma and/or APC mutated melanoma. The cancer may exclude cancers having one or more BRAF mutations, for example, BRAF mutated melanoma, NSCLC and NF1. The cancer may be colorectal cancer (CRC), for example, AXIN1 mutated CRC and/or APC mutated CRC. In particular, the cancer may be consensus molecular subtype 2 (CMS2) CRC. The cancer may be a cancer that does not have a mutation in the RAS/MAPK pathway. The cancer may be a cancer that is non-responsive to treatment with chemotherapies and/or immunotherapies. The cancer may be a cancer that is resistant or refractory to treatment with chemotherapies and/or immunotherapies.

The MEK inhibitor or a pharmaceutically acceptable salt thereof may be provided as a first line or a second (or subsequent) line treatment. When the MEK inhibitor is a second (or subsequent) line treatment, the cancer may be refractory (did not respond to the prior line of therapy) or relapsed (initially responded to the prior line of therapy but exhibited reduced or no efficacy). For example, the MEK inhibitor or a pharmaceutically acceptable salt thereof may be provided as a second line treatment in post-sorafenib treated cancer (e.g. HCC) subjects. As such, the subject may have been treated with sorafenib prior to treatment with the MEK inhibitor or pharmaceutically acceptable salt thereof. The MEK inhibitor or a pharmaceutically acceptable salt thereof may be provided as a second line treatment in post-lenvatinib treated cancer (e.g. HCC) subjects. As such, the subject may have been treated with lenvatinib prior to treatment with the MEK inhibitor or pharmaceutically acceptable salt thereof. The subject may have been non-responsive to the first line treatment.

The method may include a step of evaluating a subject suffering from cancer to identify the genetic or epigenetic makeup of cancer cells of the subject. In particular, tumours in cancer subjects may be systematically surveyed to identify the underlying somatic genetic changes in sequence, expression, and copy number. Specifically, the method may include a step of analysing one or more tumours or cancer cells of the cancer subject for the presence of one or more mutations in AXIN1 and/or APC. The mutational status of AXIN1 and/or APC in cancer cells may be routinely tested to predict the response of a patient to treatment with a MEK inhibitor and thus assist a clinician in deciding on the best treatment for the patient.

### Combination Treatments

Treatment (i.e. the MEK inhibitor or a pharmaceutically acceptable salt thereof) may be combined with one or more additional treatments for cancer. As such, the method may further comprise a step of administering an additional treatment for cancer to the subject. The MEK inhibitor or a pharmaceutically acceptable salt thereof and the additional treatment for cancer may be administered simultaneously, sequentially or separately. The MEK inhibitor or a pharmaceutically acceptable salt thereof and the additional treatment for cancer may be administered in combination. The MEK inhibitor or a pharmaceutically acceptable salt thereof may be administered contemporaneously, previously or subsequently to the additional treatment for cancer. The additional treatment may be any other treatment suitable for treating cancer, for example, chemotherapy, an immune checkpoint inhibitor or another RAS/MAPK inhibitor. In certain embodiments, the additional treatment is not a RAF inhibitor, for example, where the cancer is melanoma.

In particular, the additional treatment for cancer may be an immune checkpoint inhibitor, such as an inhibitor of PD-1 and/or PD-L1 or a pharmaceutically acceptable salt thereof. It is hypothesised that MEK inhibition may sensitise AXIN1 and/or APC mutated tumours to treatment with immune checkpoint inhibitors, such as inhibitors of PD-1 and/or PD-L1. PD-L1 and PD-1 respectively refer to the programmed death-ligand 1 (PD-L1) and its receptor programmed cell death protein 1 (PD-1). These are immune checkpoint proteins. PD-1 and PD-L1 inhibitors may act to inhibit the association of PD-L1 with its receptor PD-1. Accordingly, the method may further comprise a step of administering an immune checkpoint inhibitor, such as an inhibitor of PD-1 and/or PD-L1 or a pharmaceutically acceptable salt thereof, to the subject. The MEK inhibitor or a pharmaceutically acceptable salt thereof and the immune checkpoint inhibitor, such as an inhibitor of PD-1 and/or PD-L1 or a pharmaceutically acceptable salt thereof, may be administered simultaneously, sequentially or separately. The MEK inhibitor or a pharmaceutically acceptable salt thereof and the immune checkpoint inhibitor, such as an inhibitor of PD-1 and/or PD-L1 or a pharmaceutically acceptable salt thereof, may be administered in combination. The MEK inhibitor or a pharmaceutically acceptable salt thereof may be administered contemporaneously, previously or subsequently to the immune checkpoint inhibitor, such as an inhibitor of PD-1 and/or PD-L1 or a pharmaceutically acceptable salt thereof. The MEK inhibitor may be a MEK 1/2 inhibitor, for example, REC-4881 (TAK-733), or a MEK1/2 inhibitor selected from those listed above. The PD-1/PD-L1 inhibitor may be an antagonistic antibody. The PD-1/PD-L1 inhibitor may be an antagonistic monoclonal antibody. The PD-1/PD-L1 inhibitor may be selected from the group: Pembrolizumab (Keytruda), Nivolumab (Opdivo), Cemiplimab (Libtayo), Atezolizumab, Durvalumab, Avelumab, Envalfolimab, BMS-936559, CK-301, CS-1001, SHR-1316 (HTI-1088), CBT-502 (TQB-2450) and BGB-A333 or a pharmaceutically acceptable salt thereof. As used herein, the terms PD-1 inhibitor and PD-L1 inhibitor include pharmaceutically acceptable salts thereof. The immune checkpoint inhibitor may be an inhibitor of PD-1. The immune checkpoint inhibitor may be an inhibitor of PD-L1.

MEK inhibition may sensitise AXIN1 and/or APC mutated tumours to combination treatment with inhibitors of PD-1 or PD-L1. As such, the MEK inhibitor and the immune checkpoint inhibitor, such as an inhibitor of PD-1 and/or PD-L1 or a pharmaceutically acceptable salt thereof, may have a synergistic effect in the treatment of AXIN1 and/or APC mutated cancer which is greater than the additive effect of each of the MEK inhibitor and the immune checkpoint inhibitor, such as an inhibitor of PD-1 and/or PD-L1 or a pharmaceutically acceptable salt thereof, when administered separately. The therapeutically effective amount of the MEK inhibitor may be less than the amount needed to treat the AXIN1 and/or APC mutated cancer if the MEK inhibitor was administered without the immune checkpoint inhibitor, such as an inhibitor of PD-1 and/or PD-L1 or a pharmaceutically acceptable salt thereof. Similarly, the therapeutically effective amount of the immune checkpoint inhibitor, such as an inhibitor of PD-1 and/or PD-L1 or a pharmaceutically acceptable salt thereof, may be less than the amount needed to treat the cancer if the immune checkpoint inhibitor, such as an inhibitor of PD-1 and/or PD-L1 or a pharmaceutically acceptable salt thereof, was administered without the MEK inhibitor.

In certain embodiments, the additional treatment for cancer may be a RAF inhibitor. The term “RAF inhibitor” or “Raf inhibitor” as used herein refers to an inhibitor of RAF kinase, that is, any compound that downregulates, reduces or ceases RAF activity and/or function. The term “RAF inhibitor” as used herein is understood to encompass pharmaceutically acceptable salts thereof. The term “RAF inhibitor” as used herein may refer to one RAF inhibitor or a combination of two or more RAF inhibitors.

The RAF inhibitor may be any RAF inhibitor useful for the treatment of the cancer. The RAF inhibitor may be selected from the group: AAL-881, ABM-1310, Agerafenib, AP-23464, APL-102, ARQ-218, ARQ-680, ARQ-736, ARQ-761, ASN-003, AZ-304, AZ-628, B-Raf and c-RAF dual inhibitor (Redx Pharma), B-raf inhibitor (J&J), b-raf inhibitors (GSK), b-raf inhibitors (Sareum), b-raf kinase inhib (Kalypsys), B-Raf kinase inhibitors (Amgen), B-Raf kinase inhibitors (Array BioPharma), B-Raf kinase inhibitors (Array BioPharma-1), B-raf kinase inhibitors (Genentech), B-Raf kinase inhibitors (Novartis), B-raf kinase inhibitors (Pfizer-2), B-Raf kinase inhibitors (Wyeth), BAL-3833, BDTX-4933, belvarafenib, BGB-3245, BI-882370, BIIB-024, BRAF inhibitors (Astex), BRAF kinase inhibitor (Selexagen Therapeutics), c-Raf kinase inhibitors (BridgeBio), dabrafenib, Debio-0928, donafenib, DP-2514, DP-2874, DP-4978, EBI-907, EN-3352, encorafenib, FNX-006, GDC-0879, iCo-007, IKT-064, ISIS-5132, KIN-2787, L-779450, LErafAON, LErafAON-ETU, lifirafenib, lifirafenib+mirdametinib, LUT-014, LYN-00204, MCP-110, MG-D-1509, MG-D-1609, N-5355, Nanolipolee-007, naporafenib, NC-1, NCB-0594, NCB-0846, NGN-101, NMS-P285, NMS-P730, ONC-101, pan-RAF kinase inhibitors (Ipsen), pazopanib, pazopanib (SCAI), herapeutics, PF-04880594, PF-07284890, PLX-5568, PLX-8394, QLH-11906, Raf antagonists (Pfizer), raf kinase inhibitors (Astex), Raf kinase inhibitors (Novartis), raf kinase inhibitors (Telik), RAF-265, RAF-709, RAF/VEGFR2 inhibitor (Takeda), Ras/Raf/MEK/ERK inhibitors+PI3K/AKT/mTOR inhibitors (Celator), REDX-05358, regorafenib, RG-7256, RO-5126766, RO-7276389, RX-208, SAR-397769, SI-001, SJ-C1044, sorafenib, sorafenib bead (Biocompatibles), sorafenib (XSpray), STC-XXXX, substituted purines (Biogen Idec), TAK-632, tetrahydronaphthalene-derived compounds (Millennium), TL-241, tovorafenib, UAI-201, UB-941, vemurafenib, VRN-14, WYE-130600, XL-281 and ZK-261991.

### Pharmaceutical Compositions

To facilitate administration, the MEK inhibitor is, in various aspects, formulated into a physiologically-acceptable composition comprising a carrier (e.g., vehicle, adjuvant, or diluent). The particular carrier employed is limited only by physico-chemical considerations, such as solubility and lack of reactivity with the MEK inhibitor, and by the route of administration. Physiologically-acceptable carriers are well known in the art. Illustrative pharmaceutical forms suitable for injectable use include sterile aqueous solutions or dispersions and sterile powders for the extemporaneous preparation of sterile injectable solutions or dispersions (for example, see U.S. Pat. No. 5,466,468). Injectable formulations are further described in, e.g., Pharmaceutics and Pharmacy Practice, J. B. Lippincott Co., Philadelphia. Pa., Banker and Chalmers, eds., pages 238-250 (1982), and ASHP Handbook on Injectable Drugs, Toissel, 4th ed., pages 622-630 (1986)). A pharmaceutical composition comprising the MEK inhibitor is, in one aspect, placed within containers, along with packaging material that provides instructions regarding the use of such pharmaceutical compositions. Generally, such instructions include a tangible expression describing the reagent concentration, as well as, in certain embodiments, relative amounts of excipient ingredients or diluents (e.g., water, saline or PBS) that may be necessary to reconstitute the pharmaceutical composition.

Solid dosage forms for oral administration include capsules, tablets, powders, and granules. In such solid dosage forms, the active compound is admixed with at least one inert customary excipient (or carrier) such as sodium citrate or dicalcium phosphate or (a) fillers or extenders, as for example, starches, lactose, sucrose, mannitol, and silicic acid; (b) binders, as for example, carboxymethylcellulose, alginates, gelatin, polyvinylpyrrolidone, sucrose, and acacia; (c) humectants, as for example, glycerol; (d) disintegrating agents, as for example, agar-agar, calcium carbonate, potato or tapioca starch, alginic acid, certain complex silicates, and sodium carbonate; (a) solution retarders, as for example, paraffin; (f) absorption accelerators, as for example, quaternary ammonium compounds; (g) wetting agents, as for example, cetyl alcohol and glycerol monostearate; (h) adsorbents, as for example, kaolin and bentonite; and (i) lubricants, as for example, talc, calcium stearate, magnesium stearate, solid polyethylene glycols, sodium lauryl sulfate, or mixtures thereof. In the case of capsules, and tablets, the dosage forms may also comprise buffering agents. Solid compositions of a similar type may also be used as fillers in soft and hard filled gelatin capsules using such excipients as lactose or milk sugar, as well as high molecular weight polyethylene glycols, and the like.

Solid dosage forms such as tablets, dragees, capsules, pills, and granules can be prepared with coatings and shells, such as enteric coatings and others well known in the art. The solid dosage forms may also contain opacifying agents. Further, the solid dosage forms may be embedding compositions, such that they release the active compound or compounds in a certain part of the intestinal tract in a delayed manner. Examples of embedding compositions that can be used are polymeric substances and waxes. The active compound can also be in micro-encapsulated form, optionally with one or more excipients.

Liquid dosage forms for oral administration include pharmaceutically acceptable emulsions, solutions, suspensions, syrups, and elixirs. In addition to the active compounds, the liquid dosage form may contain inert diluents commonly used in the art, such as water or other solvents, solubilizing agents and emulsifiers, as for example, ethyl alcohol, isopropyl alcohol, ethyl carbonate, ethyl acetate, benzyl alcohol, benzyl benzoate, propylene glycol, 1,3-butylene glycol, dimethylformamide, oils, in particular, cottonseed oil, groundnut oil, corn germ oil, olive oil, castor oil, and sesame seed oil, glycerol, tetrahydrofurfuryl alcohol, polyethylene glycols and fatty acid esters of sorbitan, or mixtures of these substances, and the like.

Besides such inert diluents, the composition can also include adjuvants, such as wetting agents, emulsifying and suspending agents, sweetening, flavoring, and perfuming agents. Suspensions, in addition to the active compound, may contain suspending agents, as for example, ethoxylated isostearyl alcohols, polyoxyethylene sorbitol and sorbitan esters, microcrystalline cellulose, aluminum metahydroxide, bentonite, agar-agar, and tragacanth, or mixtures of these substances, and the like.

The compositions used in the methods disclosed herein may be formulated in micelles or liposomes. Such formulations include sterically stabilized micelles or liposomes and sterically stabilized mixed micelles or liposomes. Such formulations can facilitate intracellular delivery, since lipid bilayers of liposomes and micelles are known to fuse with the plasma membrane of cells and deliver entrapped contents into the intracellular compartment.

Upon formulation, solutions will be administered in a manner compatible with the dosage formulation and in such amount as is therapeutically effective. The formulations are easily administered in a variety of dosage forms such as injectable solutions, drug release capsules and the like. For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration.

### Single Agent

The MEK inhibitor may be administered as a single agent for the treatment of the cancer as described herein. In this embodiment, an additional treatment for the cancer, for example, a RAF inhibitor, is not administered. However, treatment may still be administered as a pharmaceutical composition, which will generally comprise a suitable pharmaceutically acceptable excipient, diluent or carrier. The MEK inhibitor may be administered as a single agent for the treatment of melanoma harbouring mutations in the AXIN1 and/or APC genes. The melanoma may not contain mutations in the BRAF genes.

### Subject

Typically, the terms “subject” and “patient” are used interchangeably herein. The subject is typically a mammal, more typically a human.

The subject suffering from cancer, e.g. HCC, may have a TP53 mutation in addition to the AXIN1 and/or APC mutations. The subject suffering from the cancer may have an APC mutation in addition to the AXIN1 mutation. The subject suffering from the cancer may have a TP53 mutation and an APC mutation in addition to the AXIN1 mutation. The subject may have a BRAF mutation in addition to the AXIN1 and/or APC mutation. Alternatively, the subject may not have a BRAF mutation, for example, where the cancer is melanoma.

The subject may have been non-responsive to a previous treatment for cancer, for example, a previous treatment with cabozantinib or with an immune checkpoint inhibitor. Genetic alterations in AXIN1 have shown to confer resistance to immune checkpoint inhibitors in small retrospective studies.

### Dosage and Routes of Administration

The MEK inhibitor or a pharmaceutically acceptable salt thereof may be administered in a therapeutically effective amount, this being an amount sufficient to show benefit to the subject to whom the treatment is administered. In particular, the MEK inhibitor or a pharmaceutically acceptable salt thereof may be administered at a suitable dose to provide 60-80% average pERK inhibition in the subject. The MEK inhibitor or a pharmaceutically acceptable salt thereof may be administered at a suitable dose to provide ~70% average pERK inhibition in the subject. The MEK inhibitor or a pharmaceutically acceptable salt thereof may be administered at a suitable dose to provide greater than 70% average pERK inhibition in the subject. The MEK inhibitor or a pharmaceutically acceptable salt thereof may be administered at a suitable dose to provide ~50% trough pERK inhibition in the subject. The MEK inhibitor or a pharmaceutically acceptable salt thereof may be administered at a suitable dose to provide greater than 50% trough pERK inhibition in the subject. A suitable dose may be in the range of 1-50 mg of MEK inhibitor per kg bodyweight of the subject (mg/kg). In some embodiments, the dose may be 8-16 mg/kg MEK inhibitor.

In one embodiment, the MEK inhibitor is REC-4881 or a pharmaceutically acceptable salt thereof and it is administered at a suitable dose to provide greater than 70% average pERK inhibition and ~50% trough pERK inhibition in the subject. The suitable dose may be in the range of 8-16 mg REC-4881 per kg bodyweight of the subject. This dose allow levels of >70% inhibition on average and ~50% inhibition at trough to be achieved.

The actual dose administered, and rate and time-course of administration, will depend on, and can be determined with due reference to, the nature and severity of the condition which is being treated, as well as factors such as the age, sex and weight of the subject being treated, as well as the route of administration. Further due consideration should be given to the properties of the treatment, for example, its in-vivo plasma life and concentration in the formulation, as well as the route, site and rate of delivery. Prescription of treatment, e.g. decisions on dosage, etc., is ultimately within the responsibility and at the discretion of general practitioners and other medical doctors, and typically takes account of the disorder to be treated, the condition of the individual patient, the site of delivery, the method of administration and other factors known to practitioners.

Dosage regimens can include a single administration, or multiple administrative doses. The treatment can further be administered simultaneously, sequentially or separately with other therapeutics and medicaments that are used for the treatment of the cancer.

The treatment may be administered to a subject in need of treatment via any suitable route. In particular, the treatment may be administered systemically. The treatment may be administered orally or parenterally by injection or infusion. Examples of preferred routes for parenteral administration include, but are not limited to, intravenous, intracardial, intraarterial, intraperitoneal, intramuscular, intracavity, subcutaneous, transmucosal, inhalation and transdermal. Routes of administration may further include enteral, for example, mucosal (including pulmonary) and rectal. The treatment may be administered via nanoparticles, microspheres, liposomes, other microparticulate delivery systems or sustained release formulations placed in certain tissues including blood.

Predicting Response to Therapy with MEK Inhibitors

The present disclosure further provides a method of using one or more mutations in AXIN1 and/or APC tumour suppressors in the WNT pathway in a subject suffering from cancer as a biomarker to evaluate the likelihood that a MEK inhibitor or a pharmaceutically acceptable salt thereof would produce an anti-cancer effect in the subject, the method comprising assaying for the presence of one or more mutations in AXIN1 and/or APC in the subject; and if one or more mutations in AXIN1 and/or APC are present in the subject, administering a MEK inhibitor or a pharmaceutically acceptable salt thereof to the subject to produce the anti-cancer effect. The one or more mutations may be loss of function mutations. The anti-cancer effect may be any effect which is of benefit to treat the subject. This includes the reduction or inhibition of the progression, severity and/or duration of the cancer or at least one symptom thereof and includes curative, alleviative or prophylactic effects.

The present disclosure therefore includes a MEK inhibitor or a pharmaceutically acceptable salt thereof for use in the treatment of a cancer having one or more mutations in AXIN1 and/or APC tumour suppressors in the WNT pathway in a subject in need thereof.

The mutational status of cancer cells and/or tumours in cancer subjects may be systematically surveyed to identify the underlying somatic genetic changes in sequence, expression, and copy number, and subjects may be treated according to the genetic or epigenetic makeup of the cancer cells.

### Definitions

Unless otherwise defined, all technical and scientific terms used herein have the meaning commonly understood by a person who is skilled in the art in the field of the present invention.

The term “treatment” as used herein and associated terms such as “treat” and “treating” means the reduction or inhibition of the progression, severity and/or duration of cancer or at least one symptom thereof. The term ‘treatment’ therefore refers to any regimen that can benefit a subject. Treatment may include curative, alleviative or prophylactic effects.

It is appreciated that certain features of the invention, which are, for clarity, described in the context of separate embodiments, may also be provided in combination in a single embodiment. Conversely, various features of the invention that are, for brevity, described in the context of a single embodiment, may also be provided separately or in any suitable sub-combination.

### EXAMPLES

### Example 1—Efficacy of MEK Inhibitor REC-4881 Versus Cabozantinib in the LI6612 AXIN1 Mutant HCC PDX Model

### Materials and Methods

Tumour Inoculation: LI6612 PDX mouse model is a liver cancer model that harbours an AXIN1 mutation and was run at Crown Biosciences in China. Tumour fragments from stock mice were harvested and used for inoculation into mice. Each mouse was inoculated subcutaneously in the right upper flank with primary human tumour xenograft model LI6612 tumour fragment (2-3 mm in diameter) for tumour development.

Randomization: The randomization started from when the mean tumour size reached approximately 170 mm³. 50 NCG mice were enrolled in the study. All animals were randomly allocated to 5 study groups. Randomization was performed based on the “Matched distribution” method. The date of randomization was denoted as day 0.

Tumour Growth Inhibition (TGI): Methods of TGI are known in the art. Tumour volumes were measured three per week after randomization in two dimensions using a caliper, and the volume was expressed in mm³ using the formula: V=(L×W×W)/2, where Vis tumour volume, L is tumor length (the longest tumour dimension) and W is tumour width (the longest tumour dimension perpendicular to L). % TGI was calculated using the formula % TGI=(TV vehicle−TV treatment)/(TV vehicle−TV initial)*100 for all mice (Wong, H, et al. Clin Cancer Res., 2012, 18 (14): 3846-3855).

Treatment Arms: 10 mice per arm were treated with either Vehicle, cabozantinib, or REC-4881 for 21 days. Cabozantinib was administered PO QD at 10 mg/kg as a suspension and REC-4881 was administered PO QD at 3 mg/kg as a suspension.

### Results

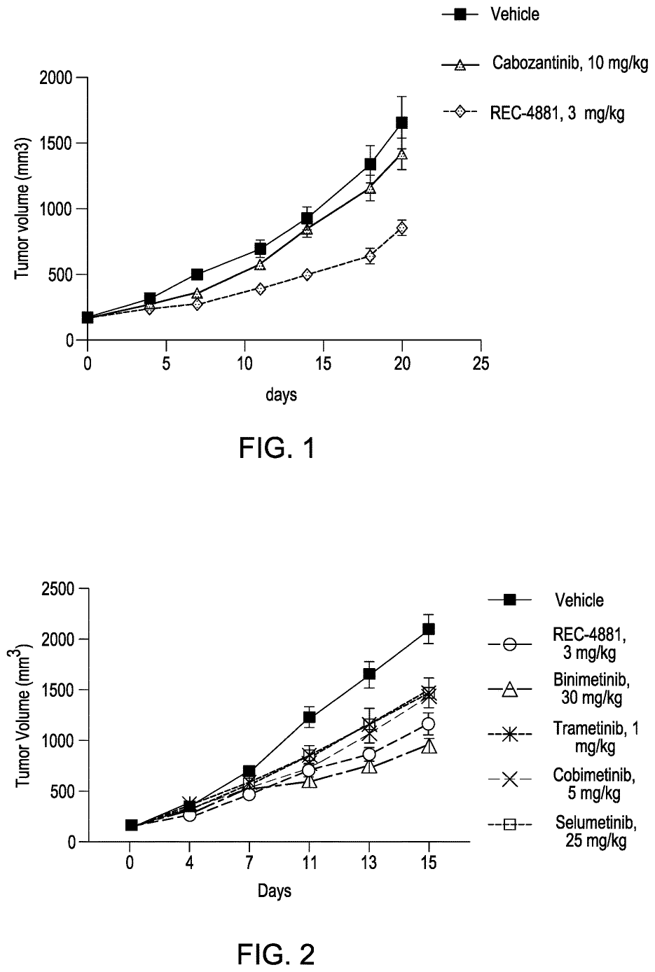

The results are shown in FIG. 1A (tumour volume) and Table 1 (Tumour Growth Inhibition (TGI). FIG. 1A shows that tumour volume decreased with treatment with REC-4881 at 3 mg/kg PO in the HCC AXIN1 mutant LI6612 PDX model and was resistant to treatment with cabozantinib. MEK inhibition was therefore superior to treatment with cabozantinib in the HCC AXIN1 mutant model.

TABLE 1
Tumour Growth Inhibition

| | | | Dose | Day | | Last | Day 20 | |
|---|---|---|---|---|---|---|---|---|
| | | Sche- | (mg/ | 0 | Last | Day | (Vol- | % |
| Test Agent | Route | dule | kg) | (n) | Day | (n) | ume) | TGI |
| Vehicle | PO | QD | 0 | 10 | 20 | 9 | 1161 | 0 |
| Cabozantinib | PO | QD | 10 | 10 | 20 | 10 | 1428 | 16 |
| REC-4881 | PO | QD | 3 | 10 | 20 | 8 | 859 | 54 |

PO—oral,
QD—once daily;
n—number of subjects;
TGI—tumor growth inhibition

### Example 2—Efficacy of MEK Inhibitor REC-4881 and 4 FDA Approved MEK Inhibitors in the LI6612 AXIN1 Mutant HCC PDX Model

### Materials and Methods

Tumour Inoculation: LI6612 PDX mouse model is a liver cancer model that harbours an AXIN1 mutation and was run at Crown Biosciences in China. Tumour fragments from stock mice were harvested and used for inoculation into mice. Each mouse was inoculated subcutaneously in the right upper flank with primary human tumour xenograft model LI6612 tumour fragment (2-3 mm in diameter) for tumour development.

Randomization: The randomization started from when the mean tumour size reached approximately 170 mm³. 60 NCG mice were enrolled in the study. All animals were randomly allocated to 60 study groups. Randomization was performed based on the “Matched distribution” method. The date of randomization was denoted as day 0.

Tumour Growth Inhibition (TGI): Methods of TGI are known in the art. Tumour volumes were measured three per week after randomization in two dimensions using a caliper, and the volume was expressed in mm³ using the formula: V=(L×W×W)/2, where V is tumour volume, L is tumour length (the longest tumour dimension) and W is tumour width (the longest tumour dimension perpendicular to L). % TGI was calculated using the formula % TGI=(TV vehicle−TV treatment)/(TV vehicle−TV initial)*100 for all mice (Wong, H, et al. Clin Cancer Res., 2012, 18 (14): 3846-3855).

Treatment Arms: 10 mice per arm were treated with either Vehicle, Binimetinib, Cobimetinib, Trametinib, Selumetinib, or REC-4881 for 15 days. Note that doses of the FDA approved MEK inhibitors may not reflect clinically relevant doses. For example, the dose of Binimetinib used in this study was 30 mg/kg BID whereas the clinically relevant dose is reported to be 2.5 mg/kg BID.

### Results

The results are shown in FIG. 2 (tumour volume) and Table 2 (Tumour Growth Inhibition (TGI). FIG. 2 shows that tumour volume decreased with treatment with MEK inhibitors REC-4881, Binimetinib, Cobimetinib, Trametinib and Selumetinib.

TABLE 2

| | | | Dose | Day | | Last | Day 15 | |
|---|---|---|---|---|---|---|---|---|
| | | Sche- | (mg/ | 0 | Last | Day | (Vol- | % |
| Test Agent | Route | dule | kg) | (n) | Day | (n) | ume) | TGI |
| Vehicle | PO | BID | 0 | 10 | 15 | 9 | 2107 | 0 |
| REC-4881 | PO | QD | 3 | 10 | 15 | 10 | 1173 | 48 |
| Binmetinib | PO | BID | 30 | 10 | 15 | 10 | 961 | 59 |
| Cobimetinib | PO | QD | 5 | 10 | 15 | 10 | 1455 | 34 |
| Trametinib | PO | QD | 1 | 10 | 15 | 9 | 1475 | 33 |
| Selumetinib | PO | BID | 25 | 10 | 15 | 9 | 1477 | 33 |

PO—oral,
BID—twice daily;
QD—once daily;
n—number of subjects;
TGI—tumor growth inhibition

### Example 3—Efficacy of MEK Inhibitor REC-4881 Single Agent and in Combination with Anti-PD-1 in a B16F10-Ova Melanoma Syngeneic Model (with APC Mutation) in Female C57BL/6 Mice Materials and Methods

Cell Culture: The B16F10-OVA tumour cells were maintained in vitro with RPMI1640 supplemented with 10% fetal bovine serum at 37° C. (+1 ug/ml puromycin) in an atmosphere of 5% CO₂ in air. The cells in the exponential growth phase were harvested and quantitated by cell counter before tumour inoculation.

Tumour Inoculation: Each mouse was inoculated subcutaneously at the right lower flank region with B16F10-OVA tumour cells (2×10⁵) in 0.1 ml of PBS for tumour development.

Randomization: Randomization started when the mean tumour size reached 81 mm³. A total of 40 mice were randomly enrolled in the study and allocated into 4 groups, with 10 mice for each group. Randomization was performed based on “Matched distribution” method (Study Director TM software, version 3.1.399.19). Dosing was implemented right after randomization on Day 10, 10 days after tumour inoculation.

Tumour Growth Inhibition (TGI): Methods of measuring TGI are known in the art. Tumour volumes were measured three per week after randomization in two dimensions using a caliper, and the volume was expressed in mm³ using the formula: V=(L×W×W)/2, where Vis tumour volume, L is tumour length (the longest tumour dimension) and W is tumour width (the longest tumour dimension perpendicular to L). % TGI was calculated using the formula % TGI=(TV vehicle−TV treatment)/(TV vehicle−TV initial)*100 for all mice (Wong, H, et al. Clin Cancer Res., 2012, 18 (14): 3846-3855).

Treatment Arms: 10 mice per arm were treated with either Anti-PD-1, REC-4881, or the combination of REC-4881+Anti-PD-1 for 13 days. REC-4881 was administered at 3 mg/kg PO QD for 13 total doses while Anti-PD-1 was administered at 10 mg/kg IP BIW for a total of 4 doses.

### Results

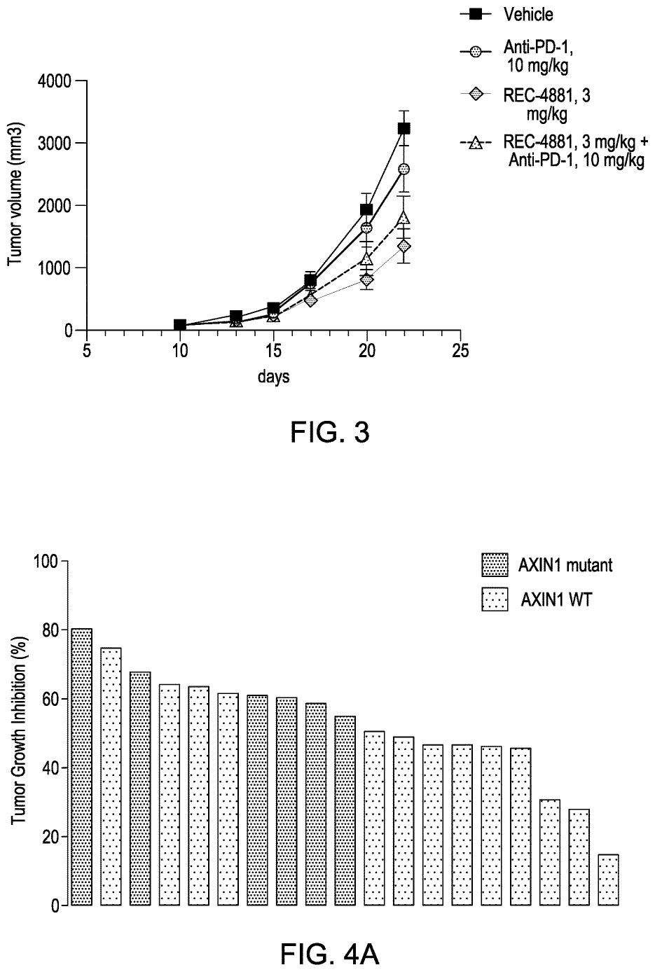

The results are shown in FIG. 3 (tumour volume) and Table 3 (Tumour Growth Inhibition (TGI)). FIG. 3 shows that tumour volume decreased with treatment with MEK inhibitor REC-4881, Anti-PD-1 alone or both treatments combined.

TABLE 3

| | | | Dose | Day | | Last | Day 15 | |
|---|---|---|---|---|---|---|---|---|
| | | Sche- | (mg/ | 0 | Last | Day | (Vol- | % |
| Test Agent | Route | dule | kg) | (n) | Day | (n) | ume) | TGI |
| Vehicle | PC | QD | 0 | 10 | 13 | 10 | 3244 | 0 |
| Anti-PD-1 | IP | BIW | 10 | 10 | 13 | 10 | 2588 | 21 |
| REC-4881 | PO | QD | 3 | 10 | 13 | 10 | 1350 | 60 |
| REC-4881 + | PO + | QD + | 3 + | 10 | 13 | 10 | 1820 | 45 |
| Anti-PD-1 | IP | BIW | 10 | | | | | |

PO—oral,
IP—injection;
BID—twice daily;
QD—once daily;
n—number of subjects;
TGI—tumor growth inhibition

### Example 4—Efficacy of MEK Inhibitor REC-4881 in 19 HuPrime Hepatocellular Carcinoma Xenograft Models in NCG Mice for AXIN1 Mutations Versus Non-AXIN1 Mutations

### Materials and Methods

Design: This study was run as a 3×3×3 design in a PDX mouse clinical trial (MCT) format at Crown Biosciences.

Objective: The objective of the study was to evaluate the in vivo efficacy of REC-4881 in 6 AXIN1 mutant HCC PDX models and 13 non-AXIN1 mutant HCC PDX models to determine if an association between AXIN1 mutational status and treatment response with REC-4881 was observed.

Tumour Inoculation: Tumour fragments from stock mice were harvested and used for inoculation into mice. Each mouse was inoculated subcutaneously in the right upper flank with primary human tumour xenograft model tumour fragment (2-3 mm in diameter) for tumour development.

Randomization: The randomization started from when the mean tumour size reaches approximately 100-200 mm³. 6 mice were enrolled in the study for each model. All animals were randomly allocated to 2 study groups for each model. Randomization was performed based on “Matched distribution” method. The date of randomization was denoted as day 0.

Treatment Arms: Vehicle (n=3) or REC-4881 (n=3) was administered at 3 mg/kg PO QD for up to 21 days.

Tumour Growth Inhibition (TGI): Methods of TGI are known in the art. Tumour volumes were measured three per week after randomization in two dimensions using a caliper, and the volume was expressed in mm³ using the formula: V=(L×W×W)/2, where V is tumour volume, L is tumour length (the longest tumour dimension) and W is tumour width (the longest tumour dimension perpendicular to L). % TGI was calculated using the formula % TGI=(TV vehicle−TV treatment)/(TV vehicle−TV initial)*100 for all mice. (Wong, H, et al. Clin Cancer Res., 2012, 18 (14): 3846-3855). TGI was calculated for all mice across all models at the last day of treatment. If any mouse across both Vehicle and REC-4881 arms died due to reaching humane endpoint or prior to treatment completing before 21 days, % TGI was calculated at the day all mice were alive to ensure standardization.

Data Analysis: Methods of analysing mouse clinical trials (MCTs) and estimating Progression Free Survival (PFS) from large xenograft studies are known in the art. Associations between response and treatment were analysed using use linear mixed models (LMMs) to describe MCTs as clustered longitudinal studies, which explicitly model growth and drug response heterogeneities across mouse models and among mice within a mouse model. PFS was defined as tumour volume doubling time at any point in time and obtained by linear interpolation on tumour growth data. (Guo, S., et al. BMC Cancer., 2019, 19:718. Gao, H., et al. Nat Med., 2015, 21:1318-1325). Kaplan-Meier curves calculated generated in the Python programming language with lifelines and tested for significance using the log-rank test at an alpha threshold of 0.05.

### Results

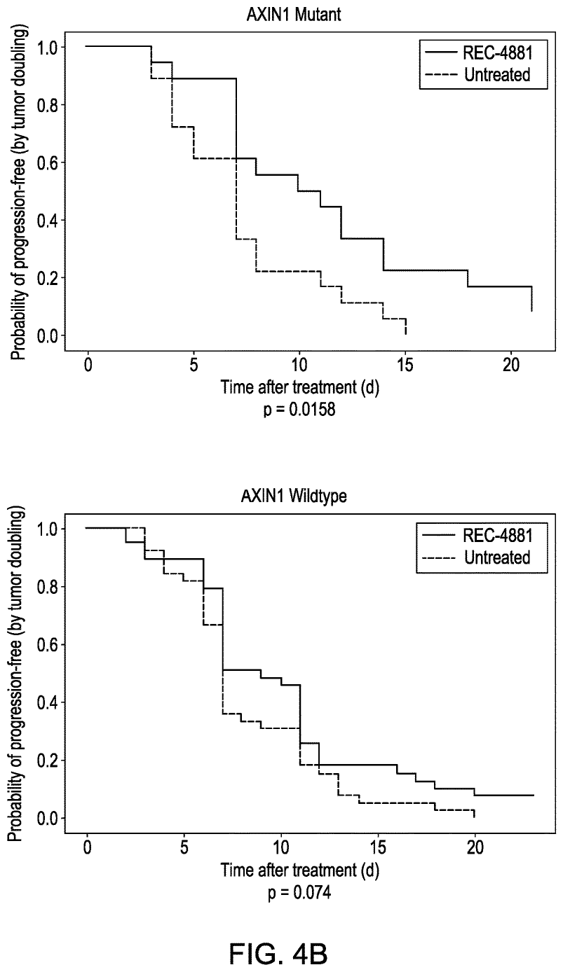

The results are shown in FIG. 4A (Tumour Growth Inhibition (TGI), FIG. 4B (probability of Progression Free Survival), and Table 4 (TGI). FIG. 4B shows that treatment of HCC with REC-4881 results in a favourable PFS benefit for models harbouring AXIN1 mutations compared to mice without AXIN1 mutations (AXIN1 Wildtype).

TABLE 4

| | | TV | TV | TV | |
|---|---|---|---|---|---|
| Model | Mutation | Vehicle | Treatment | Initial | % TGI |
| LI6692 | AXIN1 | 968 | 305 | 141 | 80 |
| LI6281 | Non-AXIN1 | 1332 | 469 | 175 | 75 |
| LI6612 | AXIN1 | 1681 | 666 | 182 | 68 |
| LI6280 | Non-AXIN1 | 3372 | 1332 | 185 | 64 |
| LI6660 | Non-AXIN1 | 794 | 370 | 122 | 63 |
| LI1005 | Non-AXIN1 | 2151 | 934 | 167 | 61 |
| LI1646 | AXIN1 | 2158 | 928 | 139 | 61 |
| LI1025 | AXIN1 | 1414 | 652 | 152 | 60 |
| LI1097 | AXIN1 | 2741 | 1243 | 171 | 58 |
| LI0334 | AXIN1 | 514 | 308 | 139 | 55 |
| LI1078 | Non-AXIN1 | 617 | 376 | 139 | 50 |
| LI1058 | Non-AXIN1 | 494 | 327 | 151 | 49 |
| LI1069 | Non-AXIN1 | 1685 | 976 | 166 | 47 |
| LI1037 | Non-AXIN1 | 3026 | 1703 | 168 | 46 |
| LI0612 | Non-AXIN1 | 1344 | 799 | 160 | 46 |
| LI0801 | Non-AXIN1 | 1974 | 1157 | 180 | 46 |
| LI0752 | Non-AXIN1 | 1926 | 1393 | 170 | 30 |
| LI6675 | Non-AXIN1 | 801 | 629 | 174 | 27 |
| LI6653 | Non-AXIN1 | 990 | 865 | 135 | 15 |

TV Vehicle—tumor volume in vehicle arm;
TV Treatment—tumor volume in treatment arm;
TV initial—tumor volume at start of study;
TGI—tumor growth inhibition

### Example 5—Efficacy of MEK Inhibitor REC-4881 in 10 HuPrime Ovarian Cancer Xenograft Models in NOD/SCID Mice

### Materials and Methods

Design: This study was run as a 3×3×3 design in a PDX mouse clinical trial (MCT) format at Crown Biosciences.

Objective: The objective of the study was to evaluate the in vivo efficacy of REC-4881 in 5 AXIN1 and/or APC mutant models and 5 non-AXIN1 and/or non-APC mutant models to determine to determine if an association between AXIN1 and/or APC mutational status and treatment response with REC-4881 was observed.

Tumour Inoculation: Tumour fragments from stock mice were harvested and used for inoculation into mice. Each mouse was inoculated subcutaneously in the right upper flank with primary human tumour xenograft model tumour fragment (2-3 mm in diameter) for tumour development.

Randomization: The randomization started from when the mean tumour size reaches approximately 100-200 mm³. 6 mice were enrolled in the study for each model. All animals were randomly allocated to 2 study groups for each model. Randomization was performed based on “Matched distribution” method. The date of randomization was denoted as day 0.

Treatment Arms: Vehicle (n=3) or REC-4881 (n=3) was administered at 3 mg/kg PO QD for up to 21 days.

Tumour Growth Inhibition (TGI): Methods of TGI are known in the art. Tumour volumes were measured three per week after randomization in two dimensions using a caliper, and the volume was expressed in mm³ using the formula: V=(L×W×W)/2, where V is tumour volume, L is tumour length (the longest tumour dimension) and W is tumour width (the longest tumour dimension perpendicular to L). % TGI was calculated using the formula % TGI=(TV vehicle−TV treatment)/(TV vehicle−TV initial)*100 for all mice. (Wong, H, et al. Clin Cancer Res., 2012, 18 (14): 3846-3855). TGI was calculated for all mice across all models at the last day of treatment. If any mouse across both Vehicle and REC-4881 arms died due to reaching humane endpoint or prior to treatment completing before 21 days, % TGI was calculated at the day all mice were alive to ensure standardization.

Data Analysis: Methods of analysing mouse clinical trials (MCTs) and estimating Progression Free Survival (PFS) from large xenograft studies are known in the art. Associations between response and treatment were analysed using use linear mixed models (LMMs) to describe MCTs as clustered longitudinal studies, which explicitly model growth and drug response heterogeneities across mouse models and among mice within a mouse model. PFS was defined as tumour volume doubling time at any point in time and obtained by linear interpolation on tumour growth data (Guo, S., et al. BMC Cancer., 2019, 19:718. Gao, H., et al. Nat Med., 2015, 21:1318-1325). Kaplan-Meier curves calculated generated in the Python programming language with lifelines and tested for significance using the log-rank test at an alpha threshold of 0.05.

### Results

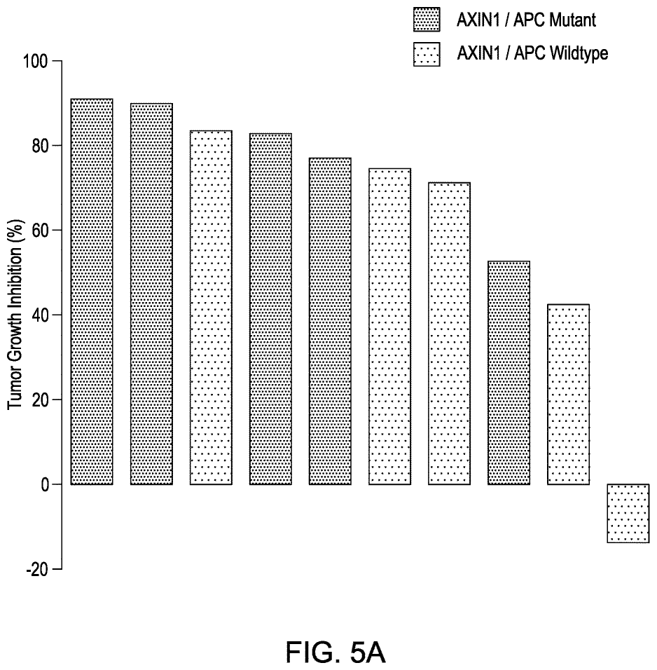

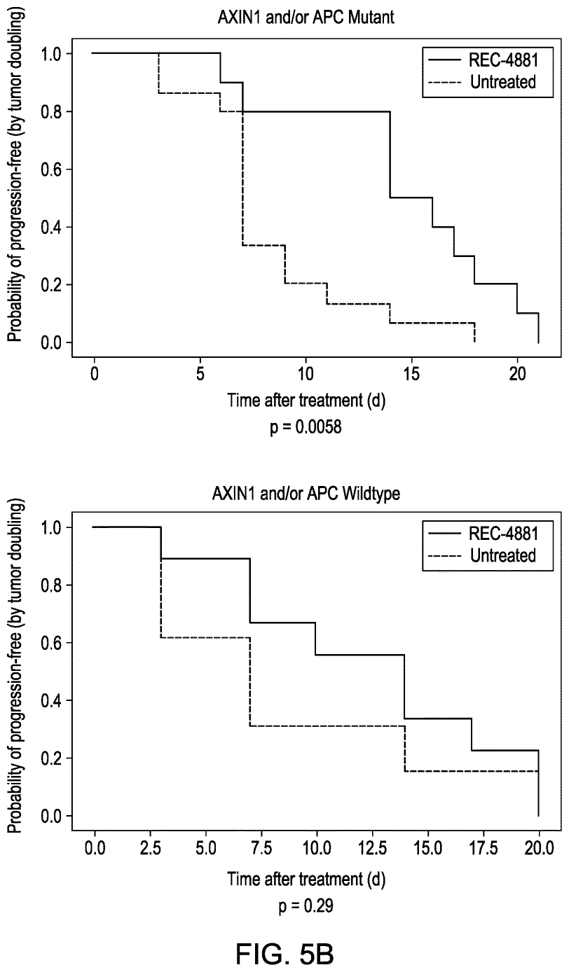

The results are shown in FIG. 5A (Tumour Growth Inhibition (TGI)), FIG. 5B (probability of Progression Free Survival) and Table 5 (TGI). FIG. 5B shows that treatment of ovarian cancer with REC-4881 results in a favourable PFS benefit for models harbouring AXIN1 and/or APC mutations compared to mice without AXIN1 and/or APC mutations (AXIN1 and/or APC Wildtype).

TABLE 5

| | | TV | TV | TV | |
|---|---|---|---|---|---|
| Model | Mutation | Vehicle | Treatment | Initial | % TGI |
| OV2034 | AXIN1/APC Mutant | 379 | 174 | 153 | 91 |
| OV1286 | AXIN1/APC Mutant | 2731 | 430 | 162 | 90 |
| OV0250 | Wildtype | 1563 | 400 | 167 | 83 |
| OV1658 | AXIN1/APC Mutant | 1560 | 402 | 155 | 82 |
| OV3708 | AXIN1/APC Mutant | 1546 | 466 | 141 | 77 |
| OV1396 | Wildtype | 613 | 272 | 149 | 74 |
| OV2015 | Wildtype | 316 | 199 | 151 | 71 |
| OV5390 | AXIN1/APC Mutant | 589 | 347 | 130 | 53 |
| OV15841 | Wildtype | 3370 | 2026 | 156 | 42 |
| OV3395 | Wildtype | 388 | 423 | 130 | −13 |

TV Vehicle—tumor volume in vehicle arm;
TV Treatment—tumor volume in treatment arm;
TV initial—tumor volume at start of study;
TGI—tumor growth inhibition

### Example 6—Combined Efficacy Analysis of MEK Inhibitor REC-4881 in 29 HuPrime PDX Mouse Models (11 AXIN1 and/or APC Mutant, 18 Non-AXIN1 and/or Non-APC Mutant)

### Materials and Methods

Methods: The data sets from both the HCC and Ovarian cancer PCTs were combined into one % TGI waterfall plot with mutational status and response correlations as well as PFS analysed similarly as previously discussed (Examples 4 and 5, FIG. 4 and FIG. 5).

### Results

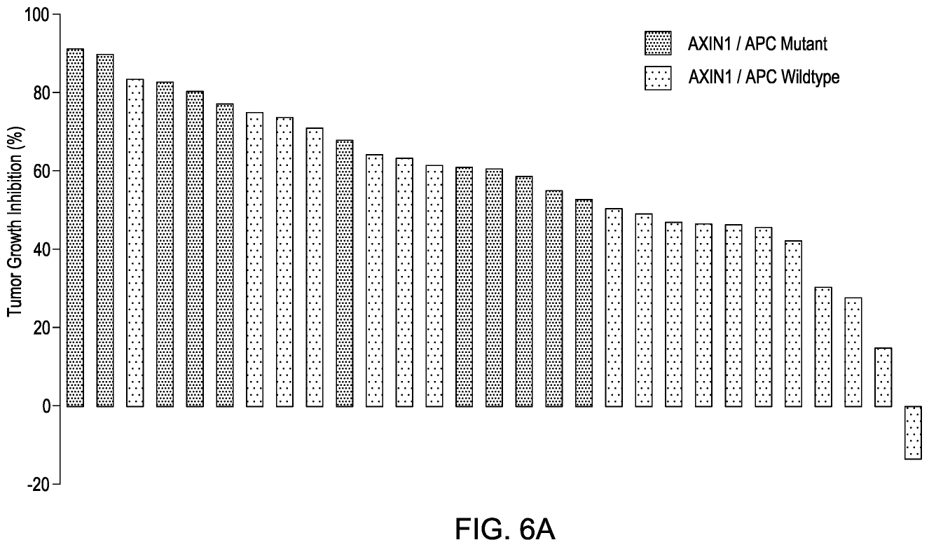

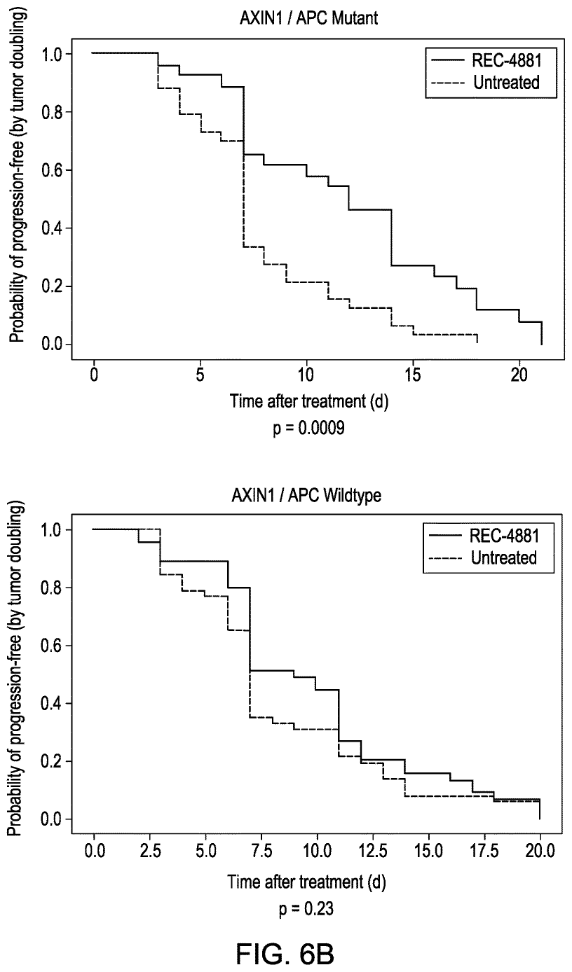

FIG. 6B demonstrates that treatment with REC-4881 results in a more significant PFS benefit in models harbouring AXIN1 and/or APC mutations when the data from HCC (FIG. 4C) and Ovarian (FIG. 5C) mouse clinical trial studies are combined.

### Example 7—Efficacy and Pharmacodynamic Assessment of MEK Inhibitor REC-4881 in a LI6692 HCC PDX Model in NCG Mice with AXIN1 Mutation

### Materials and Methods

Tumour Inoculation: LI6692 PDX model is a liver cancer model that harbours an AXIN1 mutation and from Crown Biosciences in China. Tumour fragments from stock mice were harvested and used for inoculation into mice. Each mouse was inoculated subcutaneously in the right upper flank with primary human tumour xenograft model LI6692 tumour fragment (2-3 mm in diameter) for tumour development

Randomization: The randomization started from when the mean tumour size reaches approximately 169 mm³ for efficacy study. 36 mice were enrolled in the study for efficacy study and 23 for Pharmacodynamic (PD) study. All animals were randomly allocated to 4 study groups. Randomization will be performed based on “Matched distribution” method. The date of randomization was denoted as day 0.

Efficacy Treatment Arms: 9 mice per arm were treated with either Vehicle, REC-4881 at 1 mg/kg, REC-4881 at 3 mg/kg or sorafenib at 30 mg/kg for 21 days. Both sorafenib and REC-4881 were administered as suspensions.

Pharmacodynamic Treatment Arms: 5 mice per arm were treated with REC-4881 at 1 mg/kg, REC-4881 at 3 mg/kg or sorafenib at 30 mg/kg for 2 days. Both sorafenib and REC-4881 were administered as suspensions. 8 mice were enrolled in the Vehicle arm.

Tumour Growth Inhibition (TGI): Methods of TGI are known in the art. Tumour volumes were measured three per week after randomization in two dimensions using a caliper, and the volume was expressed in mm³ using the formula: V=(L×W×W)/2, where Vis tumour volume, L is tumour length (the longest tumour dimension) and W is tumour width (the longest tumour dimension perpendicular to L). % TGI was calculated using the formula % TGI=(TV vehicle−TV treatment)/(TV vehicle−TV initial)*100 for all mice (Wong, H, et al. Clin Cancer Res., 2012, 18 (14): 3846-3855).

Pharmacodynamic Study Objective: The objective of the PD portion was to evaluate the protein expression ERK, pERK, MEK and pMEK as assessed by Western Blot and gene expression of DUSP6, SPRY4, and PPIA using SYBR Green based qPCR in the tumour samples in the LI6692 PDX models following compound treatment.

### Western Blot Procedure:

1. Load 50 μg/lane of each PDX tumour protein lysate or 20 μg/lane of each PBMC protein lysate and splenocyte lysate to pre-cast gels (26 wells, 4-15%, Bio-Rad Criterion TGX).
2. Run gels with constant voltage (60V) and stop running when the loading dye is close to the lower edge.
3. Pre-activate PVDF membranes in methanol for 2 min, followed by pre-wetting membranes and filter paper in cold transfer buffer.
4. Assemble ‘sandwich’ of filter paper—gel—membrane—filter paper and run protein transfer for about 2 hr at 280 mA.
5. When transfer is complete, immerse membranes in TBST with 5% milk, and block membranes for 1 hr at RT on a shaker.
6. Dilute primary antibodies in TBST with 5% milk at recommended dilution rate according to antibody datasheets, then incubate membranes with primary antibodies at 4° C. overnight with gentle shaking.
7. Wash membranes in TBST for 3×5 min.
8. Incubate membranes with secondary appropriate antibodies diluted in TBST with 5% milk for 1 hr at RT on a nutator.
9. Wash membranes in TBST for 3×5 min.
10. Detect target proteins with Odyssey system or Tanon 5200 chemiluminescence image analysis system.

TABLE 6
Primary Antibodies for 4 targets.

| | | MW | | | | | | Positive | Mem- |
|---|---|---|---|---|---|---|---|---|---|
| No. | Marker | (kDa) | Species | Supplier | Cat# | Dilution | Storage | control | branes |
| 1 | p-ERK | 42, 44 | Rabbit | CST | 4370 | 1:1000 | −20° C. | Hela | PVDF |
| 2 | ERK | 42, 44 | Mouse | CST | 9017 | 1:1000 | −20° C. | Hela | PVDF |
| 3 | MEK1/2 | 45 | Mouse | CST | 4694 | 1:1000 | −20° C. | Hela | PVDF |
| 4 | p-MEK | 45 | Rabbit | CST | 9154 | 1:1000 | −20° C. | Hela | PVDF |
| 5 | GAPDH | 37 | Rabbit | CST | 5174 | 1:2000 | −20° C. | / | — |

qPCR Procedure:

### Purification of Total RNA

1. Cut tumour samples into small chunks (~30 mg).
2. Add 350 μL Buffer RLT, then, to contain the sample tube fixed to the Tissue lyser II Equipment, 30 times/s, shock for 5 minutes, and proceed to next step.
3. Add 1 volume of 70% ethanol to the lysate and mix well by pipetting. Do not centrifuge.
4. Transfer up to 700 UL of the sample, including any precipitate, to a RNeasy Mini spin column placed in a 2 mL collection tube (supplied). Close the lid, and centrifuge for 15 s at ≥13200 rpm. Discard the flow-through.
5. Add 350 μL Buffer RW1 to the RNeasy column. Close lid, centrifuge for 15s at 13200 rpm. Discard the flow-through.
6. Add 10 μL DNasel stock solution to 70 μL Buffer RDD. Mix by gently inverting the tube, and centrifuge briefly.
7. Add the DNase I incubation mix (80 μL) directly to the RNeasy column membrane and place the tubes on the bench top (20-30° C.) for 15 min.
8. Add 350 μL Buffer RW1 to the RNeasy column. Close the lid, centrifuge for 15s at 13200 rpm. Discard the flow-through.
9. Add 500 μL Buffer RPE to the spin column. Close the lid gently, and centrifuge for 2 min at 13200 rpm to wash the spin column membrane. Discard the flow-through.
10. Add 500 μL Buffer RPE to the spin column. Close the lid gently, and centrifuge for 2 min at 13200 rpm to wash the spin column membrane.
11. Place the RNeasy spin column in a new 1.5 mL collection tube (supplied). Add 50 μL RNasefree water directly to the center of the spin column membrane. Close the lid gently, and centrifuge for 1 min at full speed to elute the RNA

### RNA QC

1. The integrity of the total RNA was quantified using NanoDrop. Only high-quality RNA sample was used for future cDNA synthesis and qPCR assay

### Results

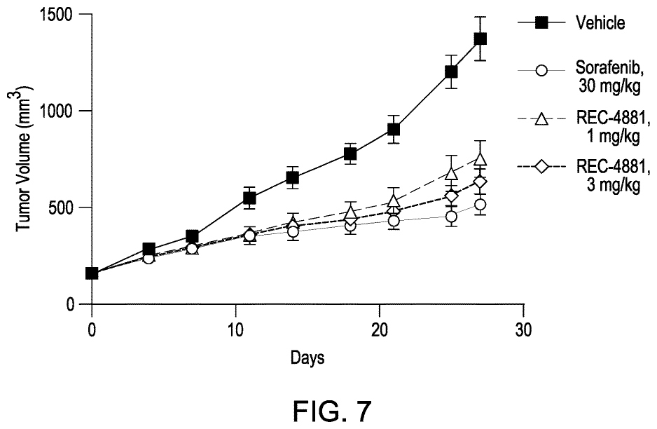

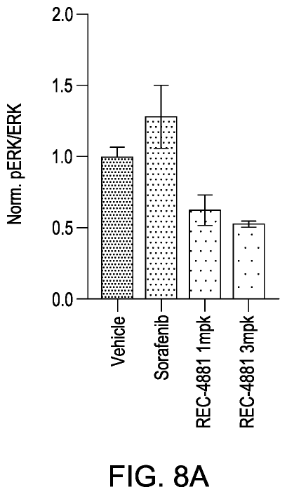

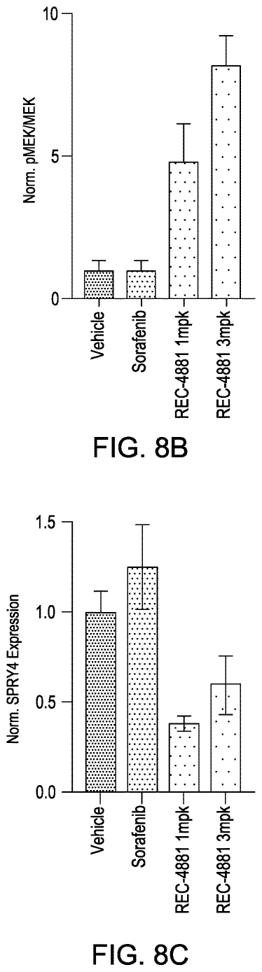

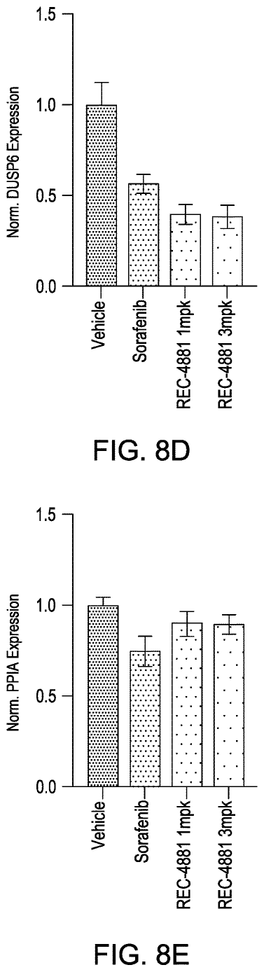

The results are shown in FIG. 7 (tumour volume) and Table 8 (Tumour Growth Inhibition (TGI). These results show that treatment with MEK inhibitor REC-4881 was non-inferior to treatment with sorafenib in mice with AXIN1 mutated HCC. FIG. 8 illustrates pharmacodynamic markers from tumour samples harvested from the in vivo PDX study (FIG. 7) demonstrating that markers of pERK (FIG. 8A), SPRY4 (FIG. 8C) and DUSP6 (FIG. 8D) are reduced two days after treatment with REC-4881 at 1 mg/kg and 3 mg/kg dosed PO with no change to PPIA (FIG. 8E) as a control. This figure demonstrates on-target and pathway engagement as well as the pathway rebound reported with MEK inhibitors with REC-4881 at 1 mg/kg and 3 mg/kg exhibiting increases in pMEK (FIG. 8B) two days after treatment.

TABLE 8

| | | | | | | | Day | |
|---|---|---|---|---|---|---|---|---|
| | | | Dose | Day | | Last | 27 | |
| Test | | Sche- | (mg/ | 0 | Last | Day | (Vol- | % |
| Agent | Route | dule | kg) | (n) | Day | (n) | ume) | TGI |
| Vehicle | PO | QD | 0 | 9 | 27 | 9 | 1385 | 0 |
| Sorafenib | PO | QD | 30 | 9 | 27 | 8 | 524 | 71 |
| REC-4881 | PO | QD | 1 | 9 | 27 | 9 | 759 | 52 |
| REC-4881 | PO | QDF | 3 | 9 | 27 | 9 | 643 | 61 |

PO—oral,
QD—once daily;
n—number of subjects;
TGI—tumor growth inhibition

### Example 9—Pharmacokinetics of MEK Inhibitor REC-4881 Following Oral Administration to Female NCG Mice

### Materials and Methods

Design: Single dose REC-4881 at 1 mg/kg and 3 mg/kg PO was administered to female NCG mice. 3 mice per treatment arm. Plasma sampling occurred at 0.5, 1, 2, 4, 8, 12, and 24 hours post dose.

Bioanalytical assay: Methods for PK analysis are known in the art. Data are presented as the mean+/−SD for each time point per treatment per mouse.

### Results

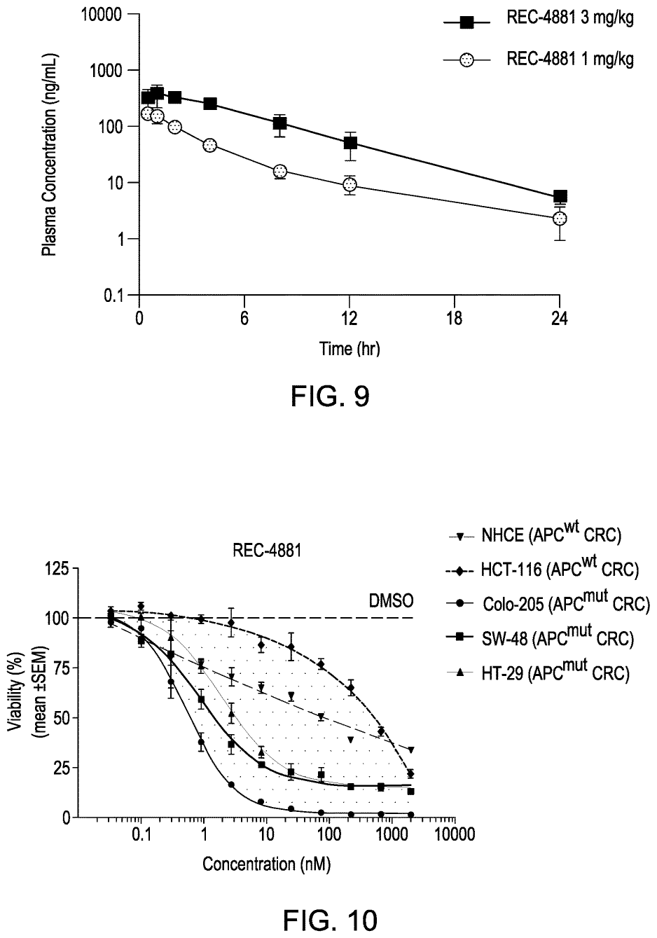

FIG. 9 demonstrates that REC-4881 can achieve clinically relevant efficacious exposures.

### Example 10—MEK Inhibition for Treatment of Colorectal Cancers with APC Mutations

### Materials and Methods

Human colorectal cancer cell lines HCT116, Colo-205, and HT29 were treated with 0.002, 0.02, 0.2 and 2 UM REC-4881 for 24 h. NHCE cells were treated with 0.37, 1.11, 3.33 and 10 UM. REC-4881 for 24 h. Cells were washed free of REC-4881 and medium, and cell viability was assayed using a CellTiter-Glo® 2.0 Cell Viability Assay (Promega; Madison, WI)

### Results

TABLE 9
EC₅₀ for Decrease in Viability in
Human Colorectal Cancer Cell Lines

| Strain | NHCE | HCT-116 | SW-48 | HT-29 | COLO-205 |
|---|---|---|---|---|---|
| REC-4881 (nM) | >2000 | >2000 | 0.86 | 2.07 | 0.54 |
| Selumetinib (nM) | >2000 | >2000 | 15.9 | 123.7 | 27.1 |

As shown in FIG. 10, MEK inhibitor REC-4881 exhibits preferentially reduced viability of APC mutant colorectal cancer cells (HT-29, SW48 and COLO-205) compared to normal human colonic epithelium (NHCE) or APC wild-type colorectal cancer cells (HCT-116), with a higher selectivity as compared with selumetinib (see Table 9). These data demonstrate that REC-4881 had well over a 1000-fold selectivity range that could be leveraged to target colorectal cancer cells harbouring APC mutations. In addition, REC-4881 exhibited greater potency and selectivity in the APC mutant lines over selumetinib (see Table 9).

### Example 11—REC-4881 Modulates Genes Downstream of the B-Catenin and Other Disease Relevant Pathways in CRCs

### Materials and Methods

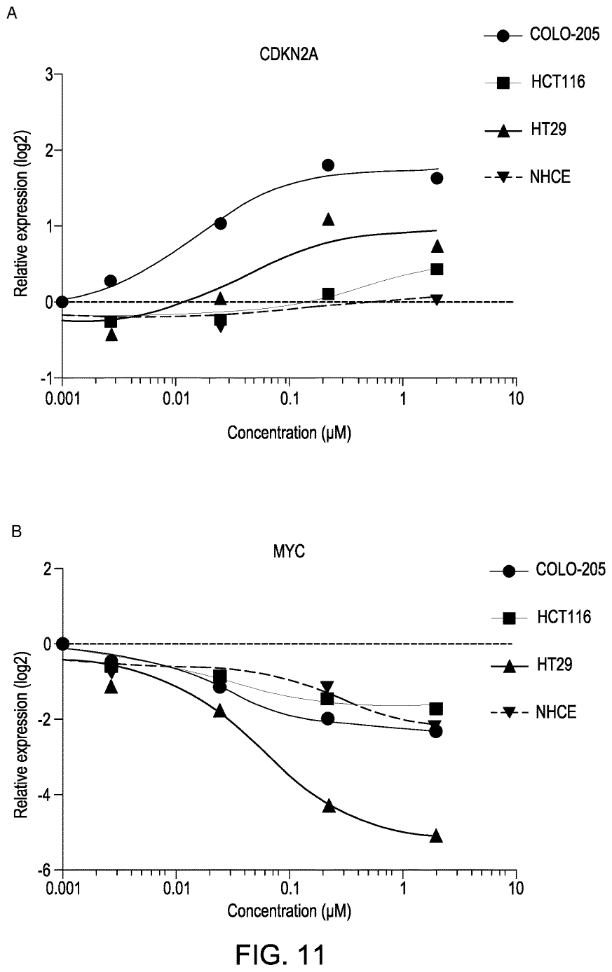

Human colorectal cancer cell lines NHCE, HCT116, Colo-205, and HT29 were treated with 0.002, 0.02, 0.2 and 2 μM REC-4881 for 24 h. NHCE cells were treated with 0.37, 1.11, 3.33 and 10 UM REC-4881 for 24 h. Cells were washed free of REC-4881 and medium, and RNA extracted. RNA was isolated and MYC (FIG. 11A) and CDKN2A (FIG. 11B) transcripts were amplified and measured using quantitative reverse transcriptase-polymerase chain reaction (qRT-PCR). Amplification of glyceraldehyde-3-phosphate dehydrogenase, hypoxanthine phosphoribosyltransferase 1, glucuronidase beta, tyrosine 3 monooxygenase/tryptophan 5-monooxygenase activation protein zeta, and TATA-box binding protein transcripts was as used as internal references for the amount of starting RNA.

### Results

As shown in FIG. 11A, REC-4881 dose-dependently increased CDKN2A in all colonic epithelial cells tested. CDKN2A gene expression was most pronounced in COLO-205 and HT-29 APC mutant cell lines. As shown in FIG. 11B, treatment of normal human colonic epithelium (NHCE), APC wild-type colorectal cancer cells (HCT-116) and APC mutant colorectal cancer cells (HT-29 and COLO-205) with REC-4881 for 24 hours resulted in a dose-dependent decrease in MYC expression. HT-29 cells treated with REC-4881 showed the largest decrease in MYC expression compared with the other CRC cell lines tested. It is hypothesised that specific contexts of APC and AXIN1 mutant cancers lead to increases in cell-cycle and MYC genes whereby MEK inhibition could reduce the activity of these downstream targets in a selective manner.

### Example 12—REC-4881 Alone or with an Anti-PD-1 in APC Mutant B16F10-Ova Syngetic Melanoma Mouse Model

Using a APC-mutant B16F10-ova syngetic melanoma mouse model, REC-4881 was administered alone or with an anti-PD1 agent to female C57BL/6 mice. Tumor samples were harvested after 13 days of treatment to the mice and assessed for CD8⁺ T-cells and T-regulatory cells. Methods: Immune cell profiling by flow cytometry. Tumor tissue was collected 4 hours post last dose. N=3 pools of three mice per group.

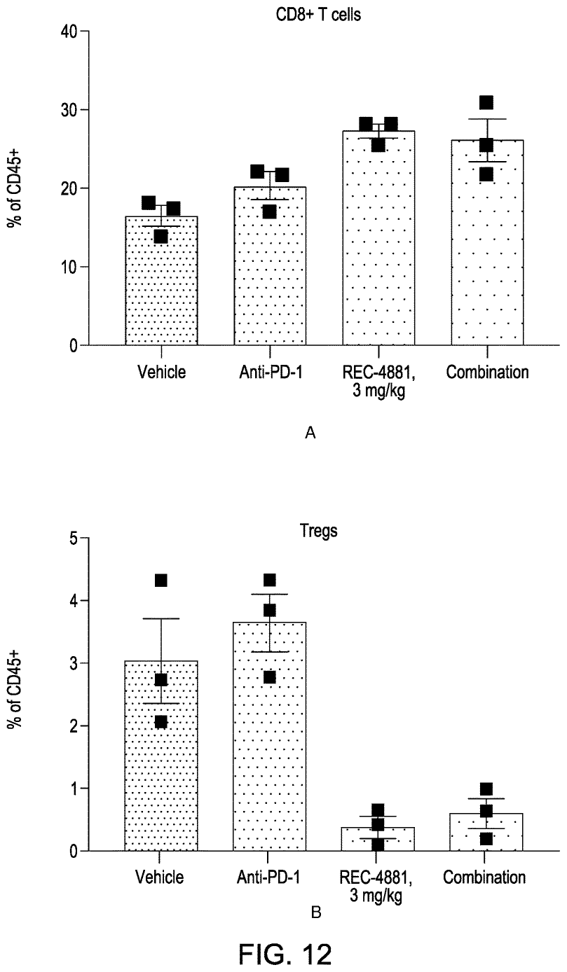

Mice administered REC-4881 alone or with an anti-PD1 agent showed increased CD8⁺ T-cells and decreased T-regulatory cells (FIGS. 12A and 12B, respectively). These data indicate that REC-4881 is able to modulate clinically relevant immune cell markers, either as a single agent or in combination with an anti-PD1.

### Example 13—REC-4881 Alone or with an Anti-PD-1 in Hepa 1-6 Synergistic Liver Hepatocellular Carcinoma (HCC) Model

Cell Culture: The Hepa 1-6 AXIN1-KO CL #1 tumor cells were maintained in vitro with Dulbecco's Modified Eagle Medium (DMEM), 10% Non-Heat-Inactivated Fetal Bovine Serum (FBS), 1% penicillin/Streptomycin/L-Glutamine (PSG).
Tumor Inoculation: Each mouse was inoculated subcutaneously at the right higher flank region with AXIN1-null tumor cells (5×10⁶) in 0.2 ml of PBS for tumor development.
Randomization: All mice were sorted into study groups based on caliper estimation of tumor burden. The mice were distributed to ensure that the mean tumor burden for all groups was within 10% of the overall mean tumor burden for the study population.
Tumor Growth Inhibition (TGI): Methods of TGI are known in the art. Tumor volumes were measured three per week after randomization in two dimensions using a caliper, and the volume was expressed in mm³ using the formula: V=(L×W×W)/2, where Vis tumor volume, L is tumor length (the longest tumor dimension) and W is tumor width (the longest tumor dimension perpendicular to L). % TGI was calculated using the formula % TGI=(TV vehicle−TV treatment)/(TV vehicle−TV initial)*100 for all mice (Wong, H, et al. Clin Cancer Res., 2012, 18 (14): 3846-3855).
Treatment Arms: 10 mice per arm were treated with either Anti-PD-1, REC-4881, Bevacizumab or the combination of REC-4881+Anti-PD-1, or Bevacizumab+Anti-PD-1 for 19 days. REC-4881 was administered at 1 mg/kg or 3 mg/kg PO QD for 19 total doses, Anti-PD-1 was administered at 10 mg/kg IP BIW for a total of 6 doses, and Bevacizumab was administered at 10 mg/kg IP BIW for a total of 6 doses.
Western Blot: Methods of western blot are known in the art. Primary antibodies: Axin (C76H11) rabbit mAb #2087 and GAPDH (D16H11) XP® rabbit mAb #5174. Diluted 1:1000 in blocking buffer Secondary antibody: Anti-rabbit IgG, HRP-linked antibody #7074. Diluted 1:5000 in blocking buffer ECL image 5 min exposure Predicted MWs: Axin-1 110 kDa, GAPDH 37 kDa

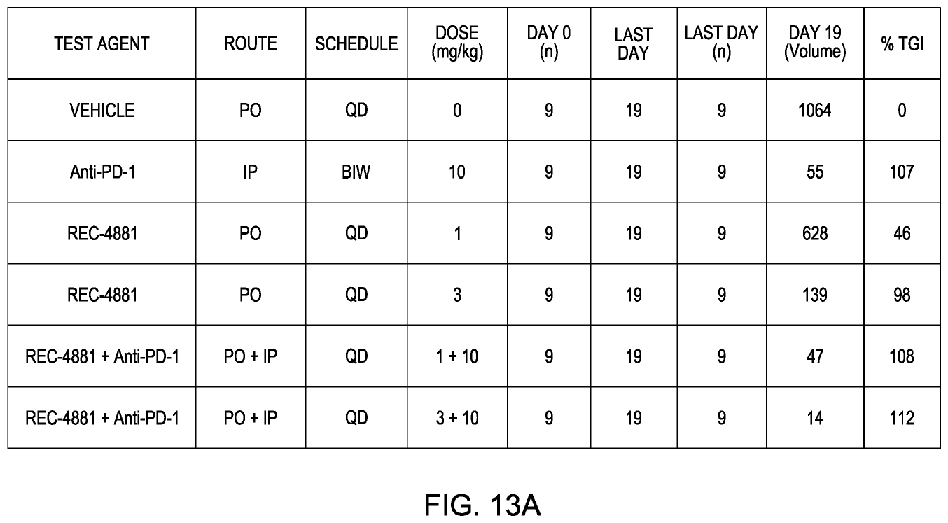

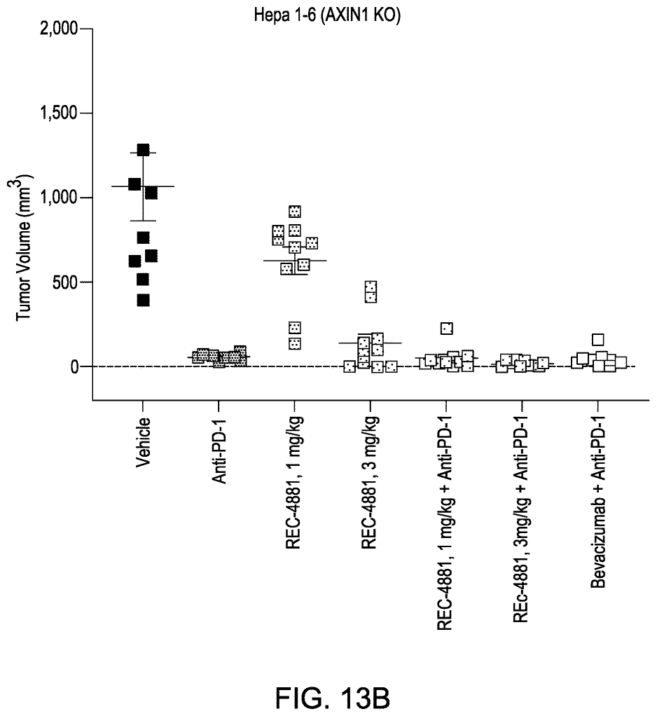

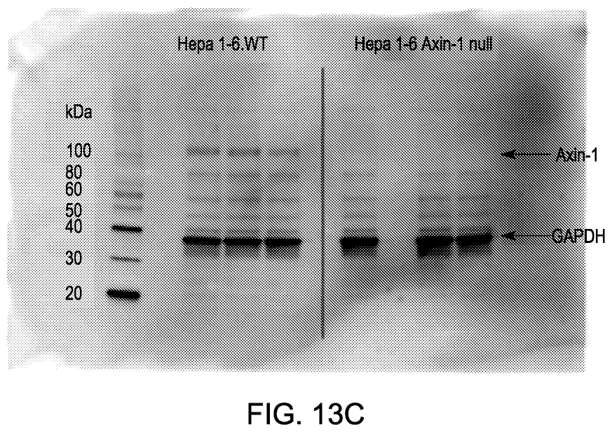

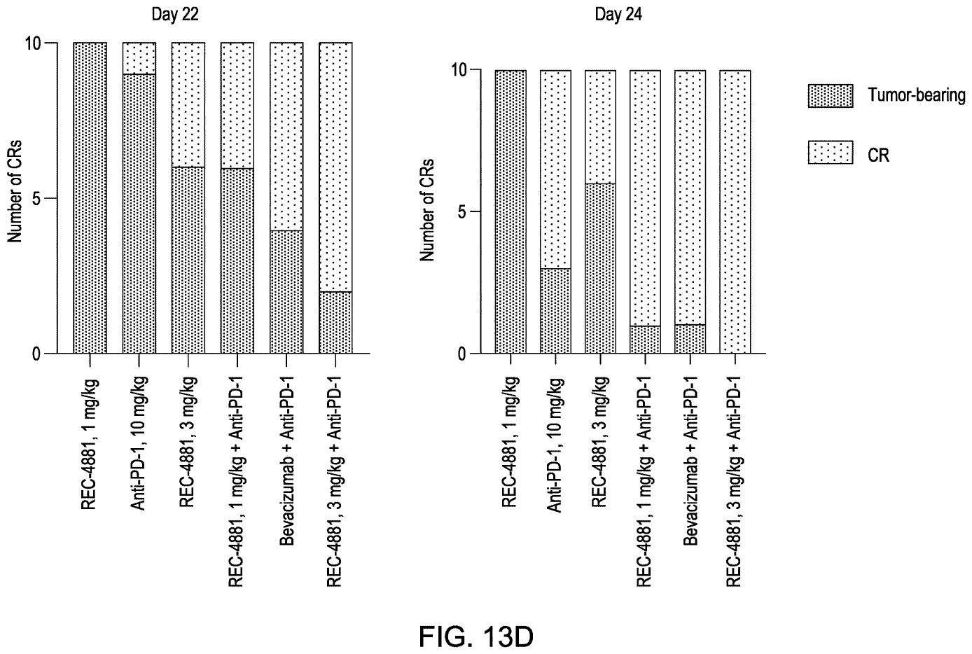

REC-4881 was administered at 1 mg/kg and 3 mg/kg orally either as a single agent or in combination with anti-PD-1 at 10 mg/kg IP a Hepa 1-6 syngeneic liver model (FIG. 13A and FIG. 13B), which was engineered to KO AXIN1 (FIG. 13C). This figure demonstrates that REC-4881 has potent single agent activity and may result in a faster response (FIG. 13D) when combined with anti-PD-1 in this model.

1. A method of treating a cancer having one or more mutations in AXIN1 and/or APC in a subject in need thereof, the method comprising:
administering a therapeutically effective amount of a MEK inhibitor or a pharmaceutically acceptable salt thereof to the subject to treat the cancer.

2. The method of claim 1, wherein the MEK inhibitor is selected from the group: REC-4881, Binimetinib, Cobimetinib, Selumetinib and Trametinib, or a pharmaceutically acceptable salt thereof.

3. The method of claim 2, wherein the MEK inhibitor is REC-4881 or a pharmaceutically acceptable salt thereof.

4. The method of claim 1, wherein the cancer is selected from the group: hepatocellular carcinoma, colorectal, liver, bladder, endometrial, melanoma, ovarian, lung, pancreatic and gastric cancers.

5. The method of claim 4, wherein the cancer is hepatocellular carcinoma.

6. The method of claim 4, wherein the cancer is ovarian cancer.

7. The method of claim 4, wherein the cancer is melanoma.

8. The method of claim 4, wherein the cancer is colorectal cancer.

9. The method of claim 8, wherein the colorectal cancer is classified as CMS2 colorectal cancer.

10. The method of claim 1, wherein the method further comprises a step of evaluating a subject suffering from cancer for the presence of one or more mutations in AXIN1 and/or APC.

11. The method of claim 1, wherein the method further comprises a step of administering one or more additional treatments for the cancer to the subject.

12. The method of claim 11, wherein the one or more additional treatments comprise treatment with an immune checkpoint inhibitor.

13. The method of claim 12, wherein the immune checkpoint inhibitor is an inhibitor of PD-1 and/or PD-L1 or a pharmaceutically acceptable salt thereof.

14. The method of claim 13, wherein the MEK inhibitor is REC-4881 or a pharmaceutically acceptable salt thereof.

15. The method of claim 11, wherein the one or more additional treatment comprise treatment with a RAF inhibitor.

16. The method of claim 1, wherein the MEK inhibitor or pharmaceutically acceptable salt thereof is provided as a second line treatment wherein the subject was previously subjected to a first line treatment for the cancer with an immune checkpoint inhibitor and wherein the subject was non-responsive to the treatment with the immune checkpoint inhibitor.

17. The method of claim 1, wherein the cancer is relapsed or refractory.

18. The method of claim 1, wherein the MEK inhibitor or pharmaceutically acceptable salt thereof is administered as a single agent.

18-19. (canceled)

20. A method of using one or more mutations in AXIN1 and/or APC in a subject suffering from cancer as a biomarker to evaluate the likelihood that a MEK inhibitor or a pharmaceutically acceptable salt thereof would produce an anti-cancer effect in the subject, the method comprising:
assaying for the presence of one or more mutations in AXIN1 and/or APC in the subject; and
if one or more mutations in AXIN1 and/or APC are present in the subject, administering a MEK inhibitor or a pharmaceutically acceptable salt thereof to the subject to produce the anti-cancer effect.

21. The method of claim 1, wherein the MEK inhibitor or pharmaceutically acceptable salt thereof is administered to the subject at a suitable dose to provide greater than 70% average pERK inhibition and ~50% trough pERK inhibition in the subject, and/or in the range of 8-16 mg of MEK inhibitor per kg bodyweight of the subject.
